(19) United States
(12) Patent Application Publication
(10) Pub. No.: US 2014/0179564 A1
(43) Pub. Date: Jun. 26, 2014

(54) COMPOSITIONS AND METHODS FOR SELECTION OF NUCLEIC ACIDS CONTAINING MODIFIED BASES

(71) Applicant: Pacific Biosciences of California, Inc., Menlo Park, CA (US)

(72) Inventors: Jonas Korlach, Newark, CA (US); Tyson A. Clark, Menlo Park, CA (US); Stephen Turner, Kirkland, WA (US)

(73) Assignee: Pacific Biosciences of California, Inc., Menlo Park, CA (US)

(21) Appl. No.: 14/068,293

(22) Filed: Oct. 31, 2013

Related U.S. Application Data
(60) Provisional application No. 61/721,206, filed on Nov. 1, 2012.

(51) Int. Cl. C12Q 1/68

(52) U.S. Cl. CPC C12Q 1/6806
USPC 506/26

(57) ABSTRACT

Methods are provided for reducing the complexity of a population of nucleic acids prior to performing an analysis of the nucleic acids, e.g., sequence analysis. The methods result in a subset of the initial population enriched for a desired property, or lacking nucleic acids having an undesired property. The methods are particularly useful for analyzing populations having a high degree of complexity, e.g., chromosomal-derived DNA, whole genomic DNA, or mRNA populations. In addition, such methods allow for analysis of pooled samples.

### CROSS-REFERENCE TO RELATED APPLICATIONS

This application claims the benefit of U.S. Provisional Application No. 61/721,206, filed Nov. 1, 2012, which is incorporated herein by reference in its entirety for all purposes. This application is further related to U.S. Provisional Application No. 61/721,339, filed on Nov. 1, 2012; U.S. Provisional Application No. 61/789,354, filed Mar. 15, 2013; and U.S. patent application Ser. No. ______, attorney docket no. 01-012702, filed on Oct. 31, 2013, all of which are incorporated herein by reference in their entireties for all purposes.

### STATEMENT REGARDING FEDERALLY SPONSORED RESEARCH

Not Applicable.

### BACKGROUND OF THE INVENTION

It is often desirable to selectively isolate molecules present in a low concentration in a sample, e.g., to facilitate analysis of such molecules without the interference of other more prevalent components of the sample. For example, in the analysis of nucleic acid sequences, actively selecting a portion of the sample nucleic acid that comprises a modified nucleotide of interest can allow a researcher to focus their analytical efforts only on those portions of the nucleic acid sample that comprise that modified nucleotide of interest. As such, the resulting “enriched” nucleic acid sample has a much higher proportion of nucleic acids having the modified nucleotide to be analyzed. Further, in some cases the concentration of a particular molecule in a sample is simply too low, rendering analysis impossible without some sort of concentration of the molecule.

Selectively enriching a sample for a molecule of interest can be performed in various ways known to those of skill in the art. For example, affinity tags have been used for purification of specific molecules of interest from a biological sample using an affinity technique. These tags are covalently or non-covalently linked to the molecules of interest. For example, an affinity tag can be incorporated into a protein of interest to form a fusion protein. The affinity tag further binds to an immobile phase, e.g., a substrate or matrix (e.g., within a column). Once bound, the substrate or matrix is washed to remove all unbound components of the sample leaving only those bound via the affinity tag. Often these tags are removable by chemical agents or by enzymatic means, such as proteolysis, which allows for removal of the selected molecules from the substrate or matrix while leaving the affinity tag behind. Once removed, the selected molecules can be further analyzed or otherwise manipulated.

With regards to isolation of specific nucleic acid sequence (“target nucleic acid”) in a complex sample (e.g., a genomic DNA sample), various methods are known in the art. Notably, “hybrid capture” methods use a nucleic acid complementary to the sequence or sequences of interest to specifically hybridize to one or more target nucleic acids. However, not all modified nucleotides have different hybridization characteristics than their counterpart unmodified nucleotides, so a complementary strand will hybridize equally or nearly equally to both. In such cases, hybridization cannot serve to distinguish between the modified and unmodified, and therefore cannot serve as a selection strategy. Accordingly, it would be desirable to provide reaction components that provide a way to not only selectively tag modified nucleotides of interest from a complex sample to allow their capture and isolation from the sample, but to also allow release of these nucleotides from the tag to facilitate subsequent manipulations, e.g., further modifications and/or analytical methods. The present invention provides these and other solutions.

### BRIEF SUMMARY OF THE INVENTION

Methods are provided for reducing the complexity of a population of nucleic acids prior to performing an analysis of the nucleic acids, e.g., sequence analysis. The methods result in a subset of the initial population enriched for a desired property, or lacking nucleic acids having an undesired property. The methods are particularly useful for analyzing populations having a high degree of complexity, e.g., chromosomal-derived DNA, whole genomic DNA, or mRNA populations. In addition, such methods allow for analysis of pooled samples.

In certain aspects, methods of generating pools of sequencing templates enriched for a type of modified base are provided. In certain embodiments, such a method comprises fragmenting a nucleic acid sample comprising the type of modified base, thereby generating a mixture comprising a first subset of nucleic acid fragments comprising the type of modified base and a second subset of nucleic acid fragments that do not comprise the type of modified base; linking adapters to nucleic acid fragments in both the first subset and the second subset; and retaining the nucleic acid fragments in the first subset from the mixture, thereby generating a pool of sequencing templates enriched for the type of modified base. In preferred embodiments, the retaining comprises binding the type of modified base to an agent linked to an affinity tag. The modified base can be a methylated or hydroxymethylated base, including but not limited to 5-methylcytosine, N⁶-methyladenosine, and 5-hydroxymethylcytosine. The adapters are preferably hairpin or stem-loop adapters that link 3′ and 5′ termini at each end of the nucleic acid fragments, and in specific implementations the adapters added to a first end of the nucleic acid fragments in the first subset are different from the adapters added to a second end of the nucleic acid fragments in the first subset. In various embodiments, the retaining comprises affinity purifying the nucleic acid fragments and/or binding driver nucleic acids to the first subset of nucleic acid fragments comprising the type of modified base. Such driver nucleic acids are typically complementary to a sequence motif that comprises the modified base in the first subset of nucleic acid fragments, and can optionally be generated from an aliquot of the nucleic acid sample. For example, driver nucleic acids can be generated by subjecting the aliquot to cleavage with a methyl-dependent restriction endonuclease, e.g., MspJI, LpnPI, FspEI, AspBHI, RlaI, or SgrTI. Subsequent to cleavage, the aliquot can be further subjected to a size selection methodology prior to binding the driver nucleic acids to the first subset of nucleic acid fragments comprising the modified base. Optionally, the driver nucleic acids can be amplified after the cleavage and prior to the binding. In preferred embodiments, driver nucleic acids comprise an affinity tag and/or a sequence motif suspected of comprising the type of modified base in the first subset of nucleic acid fragments. In certain embodiments, the binding of the driver nucleic acids to the first subset of nucleic acid fragments is performed in the presence of a strand-exchange protein. Preferably, the nucleic acid fragments in both the first subset and the second subset are not amplified prior to the retaining. Optionally, the method can further comprise removing second subset of nucleic acid fragments that do not comprise the type of modified base, e.g., by a method comprising nuclease digestion.

### BRIEF DESCRIPTION OF THE DRAWINGS

FIG. 1 illustrates an exemplary tester-driver strategy for enrichment of nucleic acids comprising a modification of interest.

FIG. 2 illustrates en exemplary embodiment of a cleavable linker for use with the methods provided herein.

FIG. 3 illustrates an exemplary strategy for enrichment of nucleic acids comprising a modification of interest.

FIG. 4 illustrates a further exemplary strategy for enrichment of nucleic acids comprising a modification of interest.

FIG. 5 illustrates an exemplary strategy for enrichment of nucleic acids comprising both a modification of interest and asymmetric adapter sequences.

### DETAILED DESCRIPTION OF THE INVENTION

### I. General

Unless defined otherwise, all technical and scientific terms used herein have the same meaning as commonly understood by one of ordinary skill in the art to which this invention belongs. Note that as used herein and in the appended claims, the singular forms “a,” “an,” and “the” include plural referents unless the context clearly dictates otherwise. Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention. Although certain preferred embodiments are described in detail herein, one of ordinary skill in the art will readily recognize the applicability of the invention in other related embodiments, e.g., enrichment of target molecules other than nucleic acid molecules.

The present invention is related to enrichment of samples for target nucleic acid molecules of interest and preferred compositions and methods for carrying out such enrichment. Such target nucleic acid molecules can comprise both natural and non-natural, artificial, or noncanonical nucleotides including, but not limited to, DNA, RNA, LNA (locked nucleic acid), PNA (peptide nucleic acid), morpholino nucleic acid, glycol nucleic acid, threose nucleic acid, and mimetics and combinations thereof. The starting population of nucleic acids can be from any source, e.g., a whole genome, a collection of chromosomes, a single chromosome, or one or more regions from one or more chromosomes. It can be derived from cloned DNA (e.g., BACs, YACs, PACs, etc.), RNA (e.g., mRNA, tRNA, rRNA, ribozymes, etc.), cDNA, or a combination thereof. The sample can be a metagenomic sample, e.g., an environmental or intestinal sample. Genomic nucleic acids can be collected from various sources including, but not limited to, whole blood, semen, saliva, tears, urine, fecal material, sweat, buccal cells, skin, and hair. The nucleic acids can be obtained from the same individual, which can be a human or other species (e.g., plant, bacteria, fungi, algae, archaea, etc.), or from different individuals of the same species, or different individuals of different species. Methods for generating a nucleic acid sample, e.g., from one of the sources listed above, is known and routine to those of ordinary skill in the art. Typically it involves cell lysis, stabilization and protection of the nucleic acids (e.g., from nuclease digestion), isolation of the nucleic acids from other components (e.g., proteins, carbohydrates, lipids, etc.) of the original sample, and optional fragmentation, e.g., by chemical, enzymatic, or mechanical means. The fragmentation serves to reduce the size of the nucleic acids, which can facilitate subsequent analyses, e.g., by providing the nucleic acids with or modifiable to have termini appropriate for subsequent steps in the analysis, e.g., cloning, ligation of adapters, circularization, and the like.

Certain aspects of the invention are particularly useful for isolating and/or enriching nucleic acids having a modification of interest, e.g., a chemical modification of one or more bases within the nucleic acid sample, a secondary structure, or an agent bound thereto. Specific examples of modifications are provided in International Patent Publication No. WO 2012/065043 A2, which is incorporated herein by reference in its entirety for all purposes. In some embodiments, such a modification comprises a methyl group or hydroxymethyl group linked to a base of a nucleic acid. In other embodiments, such a modification comprises a sugar or sugar derivative, e.g, a monosaccharide, a disaccharide, or a periodate oxidized glucose, directly or indirectly bound to a base of a nucleic acid. In yet further embodiments, a modification of interest is the result of DNA or RNA damage, e.g., 8-oxoG, pyrimidine dimer (e.g., thymine dimer or cyclobutane pyrimidine dimer), cis-platin crosslinking, oxidation damage, hydrolysis damage, photochemistry reaction products, interstrand crosslinking products, mismatched bases, and other types of “damage” to the nucleic acid. In still further embodiments, a modification of interest is the result of exposure to an agent that binds to or otherwise modifies the nucleic acid. For example, a DNA binding agent bound to a DNA molecule is considered a modification of the DNA molecule.

In certain embodiments, target nucleic acid molecules comprising one or more modifications of interest, are bound by a compound of the invention, molecules in the sample that are other than the target molecule (e.g., do not comprise the modification) are removed, and the target molecule is subsequently released into solution, thereby generating an enriched sample in which the target molecule can be subjected to various modifications and/or analyses. In other embodiments, an enrichment comprises cleavage and/or degradation of non-target nucleic acids in the sample while the target nucleic acids are protected from degradation. In preferred embodiments, at least a 10-fold, 25-fold, 100-fold, 1000-fold, or 10,000-fold molar enrichment of the target sequence of interest is achieved relative to the concentration of the target sequence in the original sample.

In certain preferred embodiments, a target molecule of interest to be isolated and/or enriched in accordance with the present invention is a nucleic acid molecule comprising at least one 5-hydroxymethylcytosine (5-hmC) base. 5-hmC is an epigenetic modification generated by TET dioxygenases that has been identified in certain mammalian cell types. To further study this modification, sensitive detection and sequencing methods are desired since traditional bisulfate sequencing methods cannot distinguish between 5-hmC and the more common modification, 5-methylcytosine (5-mC). In other preferred embodiments, a target molecule of interest to be isolated and/or enriched in accordance with the present invention is a nucleic acid molecule comprising at least one 5-fC or 5-caC, which can be present in a nucleic acid sample or can be generated by the ordinary practitioner, e.g., by treatment with mTET or other TET homologs (e.g., TET1 or TET2). In yet further preferred embodiments, a target molecule of interest to be isolated and/or enriched in accordance with the present invention is a nucleic acid molecule comprising at least sugar moiety linked thereto, which can be present in a nucleic acid sample or can optionally be generated by the ordinary practitioner, e.g., by treatment with a glucosyl transferase (e.g., beta-glucosyl transferase) as further described in Kornberg, et al. (1961) “Glucosylation of deoxyribonucleic acid by enzymes from bacteriophage-infected Escherichia coli”. J. Biol. Chem. 236: 1487-1493, which is incorporated by reference in its entirety for all purposes. Other treatments of target nucleic acids comprising modifications that can facilitate enrichment and other aspects of the methods provided herein include those described in Pastor, et al. (2011) Nature 473(7347):394-397; Song, et al. (2011) Nature Biotechnology 29:68-72; and U.S. Patent Publication No. 2011/0236894, all of which are incorporated herein by reference in their entireties for all purposes.

In certain embodiments, the invention provides methods of using an agent covalently or noncovalently bound to a modification in a strategy for enriching a pool of nucleic acids for that modification. In such embodiments, the agent specifically binds to nucleic acids comprising the modification and not to nucleic acids that lack the modification. Binding agents specific for nucleic acid modifications are known in the art and include enzymes that act on nucleic acid modifications (e.g., methyltransferases, glucosyltransferases, TET enzymes, etc.), proteins involved in DNA packaging (e.g., histone proteins), intercalating agents, antibodies to chemical modifications or bound agents, and the like. Other nucleic acid binding agents suitable for use in the methods herein are described in U.S. Patent Publication No. 20110183320, incorporated herein by reference in its entirety for all purposes. Typically, such agents are linked to affinity tags to allow separation of the bound nucleic acids from nucleic acids not bound by the agents, e.g., by immobilization (e.g., on beads or other solid surfaces) or immunoprecipitation of the agents removal of the unbound nucleic acids. In specific embodiments, the invention provides a selective chemical labeling method that not only allows linkage of an affinity tag to nucleic acids comprising 5-hmC, but also allows release of the nucleic acids once isolation and/or enrichment has been achieved. In a first step of a preferred embodiment, glucose is added to the hydroxyl group of 5-hmC using T4 bacteriophage β-glucosyltransferase (β-GT). This glucose moiety is oxidized with sodium periodate, which converts the vicinal hydroxyl groups to aldehydes. These aldehydes provide two points of conjugation with an aldehyde-reactive group, such as an amine. For example, an amine can undergo reductive amination, e.g., with sodium cyanoborohydride (NaCNBH₃), to link a molecule comprising the amine to the periodate-oxidized sugar. Aldehyde-reactive groups are known in the art and include, but are not limited to, amines, anilines, hydroxylamines, and hydrazines.

In certain aspects, a sample set of nucleic acids comprising target and non-target nucleic acids is subjected to a treatment prior to enriching for the target nucleic acids. For example, adapters can be added to all nucleic acids in the sample set prior to enriching for the target nucleic acids. In preferred embodiments, such adapters are stem-loop/hairpin adapters. In certain embodiments, the treatment serves to facilitate the subsequent enrichment, as further described below. In certain preferred embodiments, a sample set of nucleic acids is not amplified prior to enrichment and/or further analysis, e.g., sequence analysis. For certain modifications, amplification generates amplicons that lack the modification that was present in the original sample set, e.g., where a modified base has the same binding specificity to a complementary nucleotide as does an unmodified base. For example, C, 5-MeC, and 5-hmC are all complementary to G. As such, amplification of a template nucleic acid having one of these modifications using unmodified nucleoside polyphosphates will generate amplicons lacking the modification found in the original template. As such, in preferred embodiments, nucleic acids to be enriched and/or otherwise analyzed are typically not amplified in the methods herein. However, in certain embodiments, an aliquot of the original sample may be subjected to an amplification for various purposes, e.g., to provide a control sample lacking the modification or in a process to generate a driver or bait nucleic acid population, as further detailed elsewhere herein.

### II. Methods Utilizing Tester-Driver Strategies

In certain aspects, the present invention provides tester-driver strategies to enrich a nucleic acid sample for a modification of interest. As used herein, a “tester” nucleic acid population is a set of nucleic acid molecules comprising target nucleic acid molecules and non-target nucleic acid molecules. In contrast, a “driver” nucleic acid population comprises nucleic acid molecules that can bind, e.g., hybridize, to select nucleic acid molecules in the tester population, e.g., preferably either the target nucleic acids or the non-target nucleic acids in the tester population, but not both. Hybridization between the tester and driver populations followed by a selection for tester molecules that hybridize to driver molecules allows separation of target from non-target nucleic acid molecules from the tester population. The driver nucleic acids often hybridize to the target nucleic acids in the tester population, and the selection allows retention of the target nucleic acids and removal of the non-target nucleic acids. In other embodiments, the driver nucleic acids hybridize to the non-target nucleic acids, allowing removal and subsequent analysis of the “free” target nucleic acids. As such, a tester-driver strategy can comprise either a positive or negative selection, or in some cases both a positive and a negative selection can be performed, e.g., sequentially using two or more different driver populations

In certain preferred embodiments, nucleic acids in a driver population comprise a tag or other moiety to facilitate selection and/or retention of the tester-driver hybrid complexes. For example, a biotin tag can be linked to nucleic acids in the driver population so that tester-driver hybrid complexes can be captured by binding to a binding partner for biotin, e.g., streptavidin, which is bound to a solid surface, e.g., a bead or column. The bound tester-driver complexes are separated from the unbound nucleic acids, e.g., by washing, and can be eluted from the surface for further processing or analysis. Alternatively or additionally, the unbound nucleic acids that were removed from the bound complexes may be subsequently processed or analyzed. In some embodiments, the driver population is subjected to amplification prior to tester-driver hybridization and enrichment. In preferred embodiments, the tester population is not amplified before the tester-driver hybridization and enrichment.

Tags that allow the capture and, therefore, the separation of tester molecules that comprise sequence complementary to the driver from tester molecules that do not, are well known in the art and preferred tags include affinity tags, such as biotin and avidin, or a derivative thereof (e.g., streptavidin, etc.). Another type of affinity tag is an oligonucleotide complementary to a immobilized (e.g., substrate-bound) or immobilizable oligonucleotide, e.g., on a bead, column, microarray, or the like. Yet a further type of affinity tag is an antigen that binds to an immobilized or immobilizable antibody, or, alternatively, an antibody that binds to an immobilized or immobilizable antigen. Other types of affinity tags are known in the art, and specific examples of reactive functionalities for associating an affinity tag to a binding partner are provided in Table I, herein. Standard methods known to the ordinary artisan are typically used to attach a tag to a driver nucleic acid or set of driver nucleic acids. For example, many methods are known and commercially available to attach biotin molecules to nucleic acids, a process termed “biotinylation.” For example, a driver population can be subjected to PCR using a biotinylated primer and/or biotinylated nucleotides to provide amplicons that are labeled with biotin, and the label can be at a terminal position, where the primer is terminally labeled, or the label can be near an end where the primer is internally labeled. Where PCR or other amplification is used, a single round of synthesis is sometimes preferred, so that the final double-stranded molecules in the driver population have one strand that is original and only one strand that is nascent. This reduces the possibility of base changes being introduced during multi-round amplification reactions. Alternatively, a biotin dUTP label can be incorporated enzymatically, e.g, through end-labeling, nick translation, or mixed primer labeling. For example, an internal affinity tag can be linked to driver nucleic acids by subjecting the driver nucleic acids to digestion with a nicking enzyme that introduces single-strand breaks in the double-stranded driver fragments; subsequent nick translation in the presence of tagged nucleotides results in incorporation of the tagged nucleotides into the double-stranded driver fragments. The tagged nucleotides can be present in the absence or presence of the untagged nucleotides, e.g., where it is desired that the resulting driver fragments have only one or a few tags incorporated. It is also contemplated that different tagged nucleotides could be incorporated into different subpools of the driver fragments, so that at least some of the driver fragments will not have tagged nucleotides at or near their termini, which could interfere with subsequent steps, e.g., polymerase-mediated extension from a terminal nucleotide of a driver nucleic acid that is bound to a tester nucleic acid. Further, a biotin UTP analog can be enzymatically incorporated using a cleavable linker to allow removal of the biotin at a later stage in the enrichment process. Photobiotinylation can also be used to link a biotin tag to a driver nucleic acid, e.g., by using a photoactivatable biotin in the presence of UV light. Additional details on biotinylation of nucleic acids are provided in Sambrook, et al. (1989) Molecular cloning: A Laboratory Manual, 2^{nd} edition, Cold Spring Harbor Laboratory Press, NY; Coffer A, et al. Bombesin receptor from Swiss 3T3 cells, Affinity chromatography and reconstitution into phospholipid vesicles, FEBS 1990; 275(1):159-164; Ahmed, et al. Isolation and partial purification of a melanocyte-stimulating hormone receptor from B16 murine melanoma cells, A novel approach using a cleavable biotinylated photoactivated ligand and streptavidin coated magnetic beads, Biochem J 1992; 286(2):377-382; and Wahlberg, et al. (1994) Solid phase sequencing of PCR products (In: McPherson, ed. PCR II—A Practical Approach), Oxford: IRL Press, Oxford University Press; and Nelson, et al. (1991) The use of photobiotinylated PCR primers for magnetic bead-based solid phase sequencing, Human Genome III, October 21-23, San Diego, Calif., poster no. T41, all of which are incorporated herein by reference in their entireties for all purposes.

Where the tester and/or driver populations are provided as double-stranded nucleic acids, they are combined and typically denatured to allow hybridization of tester to driver. Various strategies can be used to facilitate annealing of a driver population, e.g., denaturation of the tester population prior to annealing the driver population, or use of modified nucleotides within the driver population, in particular those that enhance tester-driver hybridization, including, but not limited to, tighter binding O-methyl nucleotides, locked nucleic acids (LNAs), peptide nucleic acids (PNAs), and others known to those of skill in the art. Other examples of modified nucleotides appropriate for inclusion in a driver population are described in U.S. Patent Publication No. 2011/0183320, incorporated by reference supra. For example, where the tester is double-stranded, the driver can be provided in single-stranded form and, optionally, can comprise nucleotides that facilitate strand invasion and/or have a tighter hybridization to a single strand of the tester nucleic acid than the complementary strand has. The strand invasion can also be facilitated by addition of a strand invasion protein, such as RecA, RecT, TRF2, TFAM, or Rad51 protein. Strand invasion can be further facilitated by addition of other protein factors, e.g., single-strand binding proteins such as E. coli SSB protein.

In certain embodiments, the tester population is a population of double-stranded fragments to which stem-loop (a.k.a., “hairpin”) adapters have been added at both termini, thereby capping the ends. These tester nucleic acids therefore comprise a double-stranded tester fragment in a topologically closed construct, such that denaturation or unwinding of the double-stranded portion generates a single-stranded circular tester molecule. These molecules are beneficially used as nucleic acid sequencing templates for use in polymerase-mediated, sequencing-by-synthesis methods because they allow both strands to be sequenced repeatedly as the polymerase translocates around the topologically closed template performing “rolling-circle” synthesis. The nascent strand so generated comprises complements to both strands of the original double-stranded fragment, and where the synthesis can be monitored in real-time, the sequence of nucleotide incorporation events provides, by complementarity, the nucleotide sequence of both strands of the original tester nucleic acid. For more information on these types of sequencing templates, see, e.g., U.S. Pat. No. 8,153,375, which is incorporated herein by reference in its entirety for all purposes. Further, linking the two strands of a target nucleic acid has the added benefit of locking the two strands together since even upon strand separation the strands remain linked. It is also contemplated that the driver nucleic acids can be used as primers for subsequent analysis, e.g., amplification and/or polymerase-mediated sequencing-by-synthesis in which the target nucleic acids serve as the template nucleic acids.

In some embodiments, the driver population comprises polynucleotides having a motif that is suspected of being modified, e.g., methylated and/or glucosylated, in a tester population. For example, it may comprise one or more of the restriction-modification system recognition sequences known in the art, many of which are detailed in Wilson, G. G. (1991) Nuc. Ac. Res. 19(10):2539-2566, which is incorporated herein by reference in its entirety for all purposes. Preferably the recognition sequence comprises at least five, six, or seven specific nucleotides, but all of these need not be immediately adjacent to one another. In some cases, such recognition sequences include strings of unspecified nucleotides in between specific ones required for recognition. In some cases, such recognition sequences include alternative nucleotides, e.g., “A or G” or “C or T”. The number of specific and alternative nucleotides can be used to estimate the frequency of the recognition sequence in a sample, and therefore the amount of enrichment possible using a tester-driver strategy given a typical fragment size. For example, a recognition sequence having six specific nucleotides will occur, on average, about once every 4096 base pairs. As such, a tester population having a random distribution of the recognition sequence and consisting of primarily 1000 base pair fragments will be expected have an approximately four-fold enrichment of the recognition sequence since only about one in four fragments will be selected. Subsequent sequencing of the resulting target nucleic acids selected from the tester population by hybridization to the driver population will identify which of the selected recognition sequences were modified in the original population, and which were not, and the degree of modification for any particular locus in the population of selected nucleic acids. Although this type of selection is not directly targeting the modification itself, enrichment of a specific sequence does enrich the final composition for the modification as long as at least some of the specific sequence in the original population comprises the modification.

In certain embodiments, the driver population can be derived from the same original nucleic acid population as the tester population. For example, a small portion of the tester nucleic acid population is treated with a modification-sensitive restriction endonuclease that cleaves at a sequence suspected of being modified in the sample nucleic acid population, but only if that sequence motif does not comprise the modification. Those fragments that are modified at that sequence will not be cleaved. An exonuclease in the reaction will degrade only those molecules that were cleaved, and the molecules that were resistant to the restriction can be PCR-amplified and the amplicons used as driver molecules to select fragments from the tester population that do not comprise the specific sequence absent the modification. This selection would also select fragments that do not have the specific sequence at all, so the selection can be preceded or followed by a second selection using a driver population specific for the sequence. The final tester population would be enriched for sequences that have the modified sequence of interest. The driver amplicons having the modified sequences can also be hybridized to the tester population to create hemi-modified loci that can be selected using agents that selectively bind hemi-modified sequences, as further described elsewhere herein.

In related embodiments, the driver population can be derived from the same original nucleic acid population as the tester population, with a small portion of that population capped with hairpin or stem-loop adapters to protect the ends from exonuclease degradation. Rather than treating with a modification-sensitive restriction endonuclease, however, the driver nucleic acids are treated with one or more enzymes that render modified sites susceptible to exonuclease activity. For example, a glycosylase can be used to remove the modified base, and an AP endonuclease added to create an one-base gap where the modified base once resided. This gap provides an entry point for an exonuclease, which will degrade the fragments having a gap (i.e., the fragments that once contained a modified base), while sparing those molecules that did not have a modified base. The remaining set of nucleic acids that were not degraded can be PCR-amplified and used to capture and remove fragments from the tester population that do not contain the modifications and are therefore not desired. This is an example of a negative tester-driver selection. Optionally, this enrichment method can be preceded or followed by a second selection using a driver population specific for the sequence. The final tester population would be enriched for sequences that have the unmodified sequence of interest. The identification of both loci where a specific sequence is modified and loci where the specific sequence is unmodified, e.g., from a single organism, is a key aspect of understanding modification-based regulation of gene and protein expression, and these enrichment methods will be especially useful for mapping regions having modified and/or unmodified sequences, as described further elsewhere herein.

In specific embodiments, a driver population is generated by cleaving a nucleic acid sample with a methyl-dependent restriction endonuclease that cuts at sites that flank a methylated locus. For example, MspJI and homologs thereof cleave at fixed distances from the modification site. Modification-specific endonucleases that are homologs of MspJI include LpnPI, FspEI, AspBHI, RlaI, and SgrTI. For additional details about these endonucleases, see Cohen-Karni (2011) Proc. Natl. Acad. Sci. U.S.A. 108(27):11040-11045; and Zheng, Y. et al. (2010). Nucl. Acids Res 38:5527-5534; which are incorporated herein by reference in their entireties for all purposes. By cutting on both sides of the modifications, these proteins extract fragments of a defined length having the modification. Subsequent gel purification (or other conventional size-selection method) isolates these fragments from the non-modification-containing fragments, and the resulting mixture is enriched for modification-containing fragments. Given the length of these fragments, e.g., ˜30-35 bp, they are highly specific and therefore can be effectively used as a driver population, as described above. Preferably, a tag or other moiety to facilitate selection and/or retention of the tester-driver hybrid complexes is attached to one or both ends of the driver population, e.g., at either the 5′ or 3′ ends of one or both strands.

The driver population can also be subjected to an amplification reaction prior to introduction to a tester population, and preferably prior to adding any tagging moieties. The amplification of the driver fragments can comprise addition of noncanonical bases that enhance binding of driver to tester, either directly or indirectly. In certain preferred embodiments, the amplification is performed using nucleotides that have tighter binding to a complementary sequence in the tester population than the complementary strand in the tester population. An example of a direct enhancement is achieved by using a DNA-dependent RNA polymerase and ribonucleotides to perform the amplification, thereby producing RNA copies of the driver fragments. Since an RNA oligonucleotide binds to DNA more tightly than a DNA oligonucleotide, the resulting RNA driver fragments will have tighter binding to the tester fragments. DNA-dependent RNA polymerases (e.g., reverse transcriptases) are known to the ordinary artisan. An example of an indirect enhancement is to include uracil bases in the amplification reaction, and then subjecting the resulting amplicons to a uracil glycosylase, which will remove the uracil bases leaving abasic sites. These abasic sites can be filled with bases that binds more tightly to DNA than a canonical base, for example, a 2′-O-methyl-RNA bases, e.g., 2′-O-methyl-uridine or 2′-O-methyl-thymidine. The O-methyl-modified bases exhibit tighter binding to provide driver molecules that bind more tightly to tester molecules than unmodified driver molecules. This tighter binding can serve to stabilize the tester-driver complex and thereby facilitate enrichment of tester fragments that are complementary to the driver population. Other methods of stabilizing the tester-driver complex include addition of a strand-exchange protein, e.g., RecA, Rec T, TRF2, TFAM, or Rad 51 protein, to the driver population, preferably after the double-stranded driver has been denatured, e.g., chemically or thermally. These proteins will increase binding of the driver fragments to the complementary sequences in the tester population, and this activity is known to be enhanced by the presence of ATPγS (at least for RecA). Addition of a single-stranded DNA binding protein (e.g., E. coli SSB protein) can serve to further stabilize the tester-driver complexes.

Additionally or alternatively, the driver population can also be subjected to an amplification reaction that introduces tagging moieties into the driver amplicons. In one preferred embodiment, the amplification reaction includes biotin-labeled dUTP nucleotides, which are incorporated into the driver amplicons. Other affinity labeled nucleotides can also be used to tag the driver amplicons at internal positions. One benefit of internal labeling is that the ends of the amplicons can remain free of the tag, which could interfere with subsequent ligation or extension of the driver amplicons by template-directed nascent strand synthesis, e.g., once it is bound to a tester fragment. This internal tagging can be performed in addition to or as an alternative to the other methods of adding affinity tags to nucleic acids described elsewhere herein.

If amplification is performed, the driver population typically has adapters added to facilitate the amplification, e.g., by providing PCR primer binding sites. In certain embodiments, where these adapter sequences would interfere with hybridization of tester and driver fragments, the adapter sequences present in the resulting amplicons can be removed prior to the enrichment of the tester population. For example, in addition to the primer binding sites, the adapter sequences can include type IIs restriction sites that will remove the adapters after the amplification reaction to leave blunt ends or overhangs, and the practitioner can optionally choose the type IIs enzyme that will recreate the structure of the original driver fragments and thereby leave no residual bases from the adapter on the driver population used to enrich the tester population. There are many type IIs restriction enzymes commercially available, e.g., from New England Biolabs, and it is well within the skill of the ordinary artisan to choose one appropriate for adapters to amplify a given driver population. In other amplification strategies, hairpin adapters are added to the ends of the driver molecules to provide primer binding sites for a strand-displacing polymerase enzyme. Amplification of the driver is accomplished by rolling-circle amplification of the capped driver fragments to generate long concatemers comprising copies of both strands of the driver fragments. As these concatemers self-hybridize, the can be cleaved using an appropriate restriction endonuclease to provide double-stranded driver amplicons suitable for further use in enrichment of a tester population.

In other embodiments, adapters added to the ends of the driver fragments provide a way to lock the driver fragments onto the tester fragments. For example, adapters comprising DNA and/or RNA nucleotides can have single-stranded overhangs that are complementary to a splint oligo added to the hybridization reaction in which tester and driver fragments are annealed to each other. Since the overhangs are not complementary to the tester fragment, they remain single-stranded and available to bind to the splint oligo, which will anneal to the ends of both adapters. Annealing of the splint oligo to the ends of the adapters brings them close together so they can be ligated by a ligase enzyme, thereby creating a closed, circular driver fragment that is looped around a complementary tester fragment. This ligation of the ends of the adapters on opposite ends of a driver fragment effectively locks the driver fragment onto the tester fragment so separation cannot occur during the enrichment process. Optionally, such “locking adapters” also comprise a restriction site, preferably having a sequence motif absent from the tester population, so the locking adapters can be removed from the tester fragments after the enrichment process and before further analysis, e.g., sequencing of the tester fragments. In some embodiments, the locking adapter regions of the driver fragments comprise ribonucleotides, so they can be removed by treatment with an RNase enzyme. As such, the type of ligase enzyme used to circularize the driver molecule is dependent upon whether the terminal nucleotides in the locking adapters are DNA or RNA nucleotides.

In yet further embodiments, an adapter added to an end of the driver fragments provides a way to select for the driver fragments bound to the tester fragments. For example, the adapter can comprise a sequence complementary to an oligonucleotide that is immobilized on a solid surface, such as a magnetic bead, column, planar surface, etc. Once the tester-driver hybrid molecules are formed, the immobilized oligonucleotides are added, anneal to the driver adapters, and thereby capture the tester-driver complexes.

In certain preferred embodiments of the invention, a driver population comprising fragments generated by cleaving a portion of a sample with a modification-dependent restriction endonuclease is used to select linear fragments from the sample. Kits for probe-hybridization-based enrichment of linear fragments are commercially available, e.g, the SureSelect® Target Enrichment kit (Agilent Technologies, Santa Clara, Calif.) and the SeqCap® system (Roche NimbleGen, Inc., Madison, Wis.), and may be used with the driver populations described herein, e.g., driver populations created by used of methylation-specific endonuclease digestions followed by size-selection methods (e.g., gel-based, column-based, and the like). These commercially available kits typically involve biotin-labeling of the driver fragments prior to hybridization with a tester population, followed by affinity purification of the hybrid complexes. However, in certain preferred embodiments it is desirable to modify the enriched fragments, e.g., by ligation of adapter sequences, prior to further analysis, and the methods for such modifications often require a high concentration of input nucleic acid. However, where the sequences to be enriched are a small proportion of the overall sample, the resulting pool of enriched fragments may be too small or dilute to effectively perform the desired modifications. As such, it is desirable to keep the concentration of nucleic acids high until such modifications have been completed, and to subsequently perform the enrichment process.

In certain preferred embodiments, a nucleic acid sample is generated in which the ends of the double-stranded fragments all have 3′-terminal overhangs. This can be accomplished a variety of ways, including by restriction digestion or treating the ends with a 5′ to 3′ exonuclease that leaves a 3′-terminal overhang. Restriction enzymes that produce 3′-terminal overhangs are well known and commercially available, and this digestion can be done before or after removal of the portion of the sample from which the driver population is created. However, if the exonuclease method of generating 3′-terminal overhangs is used, this should be performed after removal of the driver aliquot, and the exonuclease must be completely deactivated prior to combining the tester and driver populations. The driver aliquot is treated as described above to generate size-selected, double-stranded driver fragments comprising sequences having modifications in the sample nucleic acid. This driver population of fragments (optionally amplified) is treated to remove the 5′-terminal phosphate groups and is subsequently combined with the sample nucleic acid (tester) population, and both are denatured and allowed to anneal. The driver fragments will only hybridize to tester fragments having complementary sequences. The mixture is treated with a polymerase enzyme under conditions that promote synthesis of a nascent strand from the 3′ end of the driver fragments that are annealed to the tester fragments to provide a blunt end at the end of the tester-driver complexes that comprises the 5′ end of the tester fragment. Reannealed driver fragments that have 5′-terminal overhangs will also have those overhangs converted to blunt ends. A stem-loop adapter (“first adapter”) having an affinity tag (e.g., biotin moiety) is ligated to the blunt ends of the tester-driver complexes, but cannot be added to the reannealed driver due to the lack of 5′-phosphate groups. Reannealed tester fragments will also not receive this stem-loop adapter because of the 3′-terminal overhangs. Following ligation, any unligated first adapter is preferably removed from the mixture, e.g., by size-selection methods (e.g., electrophoretic or column-based) or addition of an exonuclease that can degrade blunt, double-stranded ends, but cannot degrade the 5′ overhang at the end of the tester-driver complex that is opposite the end linked to the first adapter; this exonuclease would also degrade any double-stranded driver fragments that comprise at least one blunt end. At this point, the only nucleic acids in the mixture comprising the first adapter having the affinity tag are the tester-driver complexes. The mixture is subsequently treated with a 3′ to 5′ single-strand exonuclease, which will degrade all 3′-terminal overhangs, both on the tester-driver complexes, as well as any reannealed double-stranded tester molecules, to create blunt ends. Since the driver fragments that hybridized to the tester fragments lacked a 5′-terminal phosphate group, the resulting tester-driver complexes now lack a 5′-terminal phosphate at the newly created blunt end. This phosphate is added to the 5′-terminal nucleotide using a kinase enzyme (e.g., T4 PNK), which will act on not only the tester-driver complexes, but also any remaining reannealed double-strand driver fragments. Following the kinase reaction, a second stem-loop adapter lacking an affinity tag is ligated to all available blunt ends, which includes not only the blunt end of the tester-driver complex, but potentially also the ends of reannealed double-stranded tester fragments and reannealed double-stranded driver fragments. This second adapter is present in excess to inhibit ligation between tester/driver complexes, tester fragments, and driver fragments. Following the second ligation reaction, the mixture is subjected to an enrichment process to capture all molecules having the affinity tag linked to the first adapter. Since only the tester-driver fragments have the first adapter, only they will be capture and retained for further analysis. Following selection, the mixture is optionally subjected to a clean-up procedure to remove any remaining unligated second adapter sequences prior to further analysis.

In a similar embodiment, both the tester population fragments and the driver populations fragments have 5′-terminal overhangs. They are mixed, denatured, and allowed to anneal together, which produces a mixture of tester-driver complexes, reannealed driver fragments, and reannealed tester fragments. The mixture is treated with a polymerase to convert all 5′-terminal overhangs to blunt ends. The only ends that won't be converted are the 3′-terminal overhangs on one end of the tester-driver complexes, which will retain the overhang. Stem-loop adapters that do not have an affinity tag are ligated to all blunt ends. These stem-loop adapters are present in excess to inhibit ligation between tester/driver complexes, tester fragments, and driver fragments. The excess, unligated stem-loop adapters are removed, e.g., using a size-selection column or gel. Subsequent treatment with an exonuclease removes the 3′-terminal overhang on the tester-driver complexes to provide a blunt end, and a stem-loop adapter comprising an affinity tag is ligated to that newly formed blunt end. Since only the tester-driver complexes will have a free blunt end to ligate to the tagged adapters, only they will be linked to the affinity tag. Subsequent affinity purification will capture the tester-driver complexes. Since the unligated affinity-tagged adapters have the affinity tag, they will also be subject to capture. As such, they can either be removed prior to capture, or after capture. Given their small size, a size-based purification is preferred, e.g., using a column- or gel-based system.

While the above-described embodiments use an affinity tag on a stem-loop adapter, alternative embodiments can instead utilize an affinity tag on the driver fragments themselves. For example, prior to addition to the tester fragments, the driver fragments are subjected to a chemical treatment to add an affinity tag at a position in the middle of the driver fragments, but that does not inhibit hybridization to the tester fragments. Method for adding an internal affinity tag are known in the art, including but not limited to amplification using tag-linked nucleotides, enzymatic incorporation, and use of photoactivatable tags (e.g., photoactivatable biotin). The driver molecules are subsequently hybridized to the tester molecules, and the 3′ end of the driver molecule is extended using a polymerase enzyme to create a blunt, double-stranded end at the 5′ end of the tester molecule. The 3′-terminal overhang at the opposite end of the tester-driver complex is degraded and stem-loop adapters are added to both ends. There is no need for two different adapters since the affinity tag is on the driver fragment hybridized to the tester fragment. Reannealed tester fragments would also receive the stem-loop adapters, but would not be linked to the affinity tags given the absence of any driver fragments. Reannealed driver fragments would have both the stem-loop adapters and affinity tags, and so would be captured with the tester-driver complexes during the enrichment procedure. However, they are much smaller than the tester-driver complexes, and so can be easily removed, e.g., by size selection after capture. This method is simpler than the one above in that the tester fragments need not have 3′-terminal overhangs, the driver fragments need not be treated to remove the 5′-terminal phosphate groups, and the tester-driver complexes need not be subjected to a kinase reaction to add a 5′-terminal phosphate. In preferred embodiments, the affinity tag can be removed prior to further analysis, e.g., by chemical cleavage, enzymatic cleavage, or photocleavage of a linker that connects it to the enriched tester-driver complexes.

In other preferred embodiments of the invention, a driver population comprising fragments generated by cleaving a portion of a sample with a modification-dependent restriction endonuclease is used to select circularized fragments from the sample. In general practice, and as discussed above, an aliquot is removed from a nucleic acid sample for use in generating the driver population. This aliquot is preferably removed after fragmenting the nucleic acid sample into long fragments, e.g., at least about 500, 1000, 2500, 5000, 10,000, 20,000, 50,000, or 100,000 base pairs long, although the aliquot can alternatively be removed prior to the fragmentation. The portion of the sample that remains after removing the aliquot is subjected to an end-repair step to ensure all ends are appropriate for ligation to stem-loop adapter sequences. The stem-loop adapter sequences are ligated to the ends of all the fragments to generate a mixture of double-stranded, closed tester fragments that are topologically circular, but structurally linear. Such molecules are described at length in U.S. Pat. No. 8,153,375, which is incorporated herein by reference in its entirety for all purposes. The aliquot from which the driver population will be derived is subjected to a digestion using a modification-dependent cutter, such as those described above, to generate double-stranded fragments (driver fragments) having sequence that is modified in the original sample. These fragments are optionally amplified, e.g., by addition of PCR adapters that are subsequently removed prior to addition of the driver population to the tester population, e.g., by restriction digestion using type IIs enzymes having recognition sequences within the adapters, as discussed at length supra. The driver fragments are linked to affinity tags and added to the population of tester fragments. The driver and tester fragments are denatured and allowed to anneal to one another. Once annealed, the affinity tag on the driver fragments is used to capture the tester-driver complexes, thereby separating them from the tester fragments that are not complementary to the driver fragments. Once the tester-driver complexes are isolated, and therefore enriched, the driver fragments can be removed, e.g., by denaturation and subsequent size-selection procedures that are standard in the art.

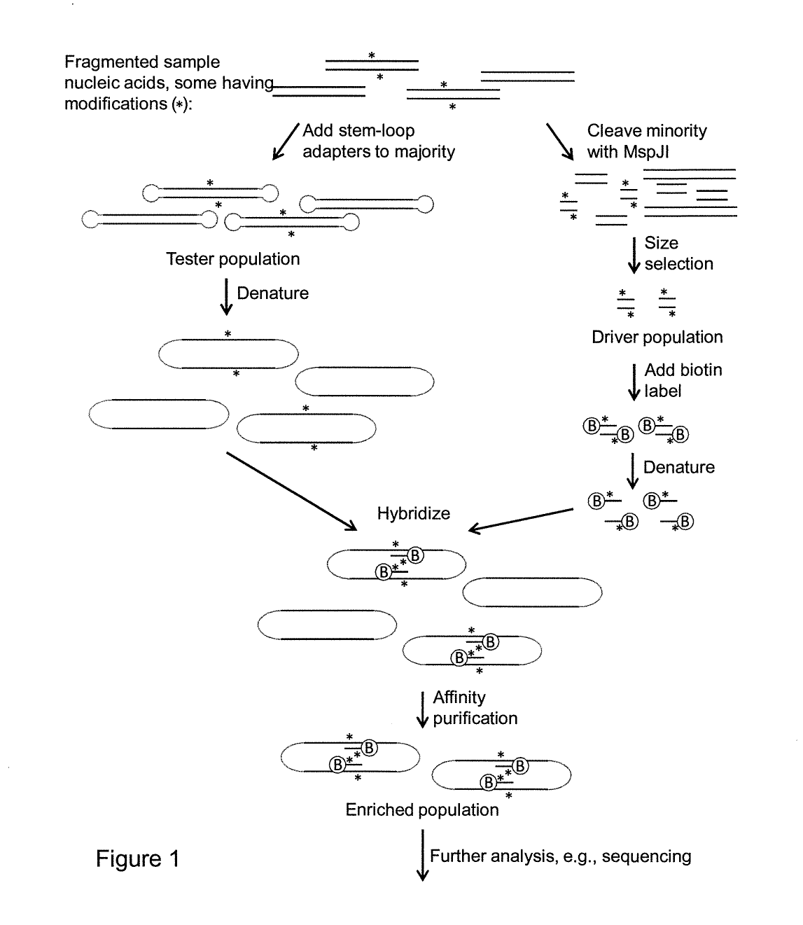

Due to the circular nature of the tester fragments, these tester-driver complexes are inherently less stable than the tester-driver complexes that comprise linear tester fragments, at least in part of the propensity for the molecules to self-hybridize and thereby displace a bound driver fragment. Having both strands of the driver fragments present in the hybridization reaction is preferred, since if both bind to a single tester molecule, it is a somewhat more stable complex since both the sense and antisense strands of the tester fragment are occupied in complementary locations. Further, where multiple different sequence motifs are present in the driver population, a single tester molecule may be bound by multiple different driver fragments in different sequence contexts. FIG. 1 provides an exemplary workflow for such an embodiment of the invention. In a first step, a fragmented sample comprising fragments with modifications and fragments lacking modifications is divided into two portions. A first portion is subjected to ligation to stem-loop adapters, and the second portion is subjected to digestion with MspJI followed by size selection to isolate a driver population. An affinity tag, biotin in this case, is added to the driver fragment population. The tester and driver populations are denatured and combined under conditions that promote hybridization, and since the driver population is double-stranded, it comprises strands complementary to both strand of the tester molecule. This tester-driver complex is affinity purified to produce a population of the tester fragments enriched for the sequences present in the driver population, i.e., sequences recognized by the methylation-specific endonuclease, MspJI.

Further methods are also contemplated for stabilizing these complexes. In some preferred embodiments, additional proteins are added to the hybridization reaction to enhance binding of driver fragments to tester fragments. For example, proteins known to enhance strand invasion or stabilize D-loops, e.g., RecA, RecT, Rad51, TRF2, or TFAM, can be added to the driver population prior to combining the driver and tester population. Such proteins have been shown to coat a single-stranded nucleic acid molecule and facilitate it's hybridization with one strand of a duplex nucleic acid while displacing the complementary strand. Proteins known to bind and stabilize a single-strand configuration can also be used to stabilize the tester-driver complex, such as E. coli SSB protein, mtSSB, and RPA protein. These single-strand binding proteins are preferably either used in combination with a protein that enhances strand invasion, and/or added after the tester-driver complexes have formed. In certain preferred embodiments, a combination of proteins known to work in vivo are used together in vitro, e.g., E. coli SSB and RecA proteins. Further information about the ability of RecA protein to perform in vitro strand pairing and invasion are provided in the literature, e.g., in Tracy, et al. (1996) Genes & Development 10:1890-1903; Yang, et al. (2012) Proc. Natl. Acad. Sci. USA 109(23):8907-8912; Handa, et al. (2012) Proc. Natl. Acad. Sci. USA 109(23):8901-8906; and Kowalczykowski, et al. (1994) Annu. Rev. Biochem. 63: 991-1043, all of which are incorporated herein by reference in their entireties for all purposes.

In certain preferred embodiments, random oligonucleotides that do not comprise the sequence motif that is modified in the tester molecules are introduced in the hybridization reaction along with the driver molecules. These random oligos will bind to the denatured, circular tester molecules, helping to keep these molecules in the single-stranded state to facilitate binding of the driver molecules and stabilization of the tester-driver complexes. Optionally, the random oligos can comprise modified bases that enhance binding to the tester molecules, e.g., 2′-O-methyl bases, PNA, LNA, and the like.

The driver molecules can also be altered or replaced with complementary molecules that have tighter binding to further stabilize the tester-driver complexes. For example, the driver molecules can be amplified using ribonucletides and a DNA-dependent RNA polymerase to generate RNA driver molecules, which will hybridize to the tester molecules more tightly than DNA driver molecules. Alternatively or in addition, locking adapters, as described supra, can be attached to the driver molecules and used to circularize the driver molecules (with the help of a splint oligonucleotide and a ligase enzyme), thereby linking them around the tester molecule to which they are hybridized.

In yet further embodiments, the stability of the circular tester-driver complexes can be enhanced by treatment with a polymerase enzyme, which will synthesize a nascent strand from the 3′ end of the driver fragment or from a primer bound to one of the stem-loop adapters at the ends of the circular tester. The synthesis of the nascent strand from a bound driver causes displacement of the complementary strand of the tester fragment, effectively increasing the footprint of the driver fragment and thereby increasing the strength with which the driver is bound, as well as decreasing the likelihood that the tester will self-hybridize and displace the extended driver. Using the bound driver as a primer requires that the driver be bound before the polymerization reaction. Alternatively, the primer is an oligonucleotide that is complementary to one or both single-stranded regions in the stem-loop adapters that cap the tester fragments. After annealing the primers, the polymerase is bound and synthesizes a nascent strand complementary to one strand of the tester fragment. In doing so, the opposite strand is displaced and rendered single-stranded. The progression of the polymerase can be controlled a number of ways, e.g., by removal of divalent cations that support the reaction, e.g., with a chelating agent; addition of divalent cations that do not support polymerization to stall the enzyme, e.g., Ca²⁺; addition of a protease to kill the enzyme; or inclusion of a blocking group on the stem-loop adapters that is upstream of the primer binding site such that it only blocks a polymerase after it has synthesized a nascent strand complementary to only one strand of the tester fragment. Various specific methods and compositions for control of polymerase reactions are more fully discussed in U.S. Pat. Nos. 8,143,030; 8,133,672; and 7,901,889, all of which are incorporated herein by reference in their entireties for all purposes. Once the nascent strand has been synthesized on a first strand of the tester fragment and the second strand of the tester fragment is single-stranded, the driver fragment can hybridize to the tester molecule and cannot be displaced by reannealing of the two strands of the tester molecule. Other proteins can also be added to further stabilize the complex, as noted above, e.g., a single-stranded DNA binding protein. Where both stem-loop adapters on the tester fragments are identical, the primers for extending from the adapter will bind to both ends. As such, a polymerase may initiate from either end of the tester, and the method is expected to provide approximately equal numbers of tester complexes having the first or second strand rendered single-stranded and available for binding to a driver strand. This is especially beneficial where the two strands of the driver fragment bind with differing affinities or strengths since the approach allows capture the tester fragments from both strands of the driver fragment. As such, the strong binding of one driver strand can compensate, at least in part, for the weak binding of the other. In some embodiments, the polymerase is a DNA-dependent RNA polymerase and the nascent strand generated is and RNA strand, which will enhance stability of the complex more than a nascent DNA strand due to the tighter binding between DNA and RNA strands as compared to between DNA strands. Yet further, following capture of the tester-driver complexes, e.g., by virtue of the affinity tag on the driver strands, the nascent strand can be degraded using an RNase, thereby restoring the original tester molecule having the two stem-loop adapters, which will self-anneal and displace the driver fragment bound thereto. Preferably, the driver fragments that are captured with the tester-driver fragments are removed, e.g., by size-selection, prior to further analysis of the captured tester fragments.

The stabilization of the tester-driver complexes can be enhanced using any of the methods described herein, alone or in combination. Further, in preferred embodiments of any of these tester-driver-based enrichment methods, the tester fragments are very long, e.g., at least about 500, 1000, 2500, 5000, 10,000, 20,000, 50,000, or 100,000 base pairs in length. This is beneficial for size-based separation of unligated adapter sequences and driver fragments from an enriched tester population. It is further useful where further analysis of the enriched population involves technologies that can analyze long nucleic acids, e.g., Single-Molecule, Real-Time (SMRT®) Sequencing by Pacific Biosciences (Menlo Park, Calif.). Being able to analyze a long nucleic acid, e.g., to generate a long, single-molecule sequence read, is important for many genomic analysis applications, e.g., analysis of highly repetitive regions, detection large structural variations, full-length cDNA sequencing, long-range haplotype phasing, and the like. Further, many of the methods described herein are focused on enrichment of nucleic acid regions having modifications, such as methylated bases. So, the methods herein, when used to enrich a population for a modification of interest, will provide invaluable information to further research in understanding how these modifications are arranged and regulated in a genome, especially where the modifications occur in highly repetitive regions or other regions that are difficult to map using short-read, second-generation technologies.

In further embodiments, a driver population comprises highly repetitive regions that are often modified, e.g., regulatory regions for gene expression. Such highly repetitive regions have been avoided by traditional nucleic acid sequence analyses due to difficulty accurately mapping such sequences with the short read lengths and biased error profiles of many first and second generation sequencing technologies. Single molecule, real-time (SMRT) sequencing (Pacific Biosciences, Menlo Park, Calif.) provides long reads and unbiased error profiles that allow accurate mapping of highly repetitive regions within a genomic sample. As such, an object of certain embodiments of the invention is to select and enrich for highly repetitive regions to allow further analysis in the absence of low repeat regions. For example, genomic regions that can be enriched by the methods provided herein include regulatory regions, heterochromatic regions, mini- and microsatellite regions, centromeric or telomeric regions, homopolymer repeat regions (e.g., triplet repeat regions), and the like. In certain preferred embodiments, one or more genes having a triplet base expansion or contraction region is enriched. A driver population can comprise polynucleotides having repeating sequence that can be used to select complementary repeating sequence from a tester population, e.g., by utilizing a tag on the driver population nucleic acids to capture driver-tester hybrid duplexes, as described supra.

### III. Reversibly Associated Affinity Tags

The ability to remove an affinity tag from a target molecule being isolated and/or enriched allows recovery of the target molecule dissociated from the affinity tag. This is a benefit, e.g., when the affinity tag is bound to a solid surface or phase (e.g., bead, substrate, column, etc.) and further analysis of the target molecule requires transfer to a liquid phase. Reversibly associated affinity tags are especially suitable where an affinity tag linked to the target molecule becomes covalently linked to a binding partner in the solid phase. Typically, after the affinity tag is associated with its binding partner in the solid phase, the solid phase is washed to remove “non-target” components of a sample, which are not linked to the affinity tag, thereby isolating the target molecules of interest from the sample. Since covalent linkages are more stable than non-covalent linkages, a covalent interaction between an affinity tag and its binding partner can reduce the amount of affinity tag-linked target molecule that is lost during the washing of the solid phase.

In preferred embodiments, the invention provides a linker molecule comprising an affinity tag, a cleavable portion, and a reactive group that specifically binds to the target molecule. In certain embodiments, the reactive group is an aldehyde-reactive group is positioned at the end of the linker molecule, e.g., at the end opposite the affinity tag. Such a linker molecule is also termed a “reversibly associated affinity tag” because the cleavable portion allows removal of the portion of the linker molecule comprising the affinity tag, e.g., to release it from the target molecule at a time when it is no longer desired. For example, a disulfide bond within the linker molecule between the affinity tag and the aldehyde-reactive end is an especially preferred configuration. Other cleavable portions can comprise, for example, a photocleavable moiety (e.g., from Ambergen, Inc. (Watertown, Mass.) or a chemistry that can be cleaved under mild conditions. For example, a cleavable linker comprising a diazobenzene derivative that is cleavable under mild reducing conditions is described in Verhelst, et al. (Angew. Chem. Int. Ed. 2007, 46:1-4), which is incorporated in its entirety for all purposes. Photocleavable moieties are known in the art, e.g., in Bai, et al. (Nucleic Acids Res. 2004, 32(2):535-541) and Piggott, et al. (Tetrahedron Letters 2005, 46:8241-8244), both of which are incorporated herein in their entireties for all purposes; and are commercially available, e.g., from Ambergen.

Many types of affinity tags can be used in the linker molecules of the invention. Preferred affinity tags that covalently and non-covalently associate with their binding partner include, but are not limited to, those shown in Table I below. While covalent interactions are preferred, highly stable non-covalent interactions are also contemplated for use with the methods herein. Stable, non-covalently associating binding pairs can include, but are not limited to, antibodies that stably bind their antigens and biotin (which binds to avidin and streptavidin).

Synthesis of a reversible affinity tag of the invention typically begins with an N-hydroxysuccinimide ester linked to a tag that can be used for affinity purification. As noted above, the increased stability of covalent interactions as compared to non-covalent interactions provides a benefit of increased recovery of a tagged molecule of interest. Cleavable linkers to attach an affinity tag to a binding agent that interacts with a modification can be based on various chemistries, e.g., 9-fluorenylmethoxycarbonyl (Fmoc), (tert)-butyloxycarbonyl (Boc or tBoc), cystamine linkers, or others known in the art. In particular, Fmoc-based linker cleavage involves a mild base treatment, but construction of the linker requires considerable biochemical synthesis work. Use of cystamine linkers typically involves reductive amination with NaCNBH₃ to link to the affinity tag, e.g., biotin. This reaction is well known and allows conjugation with any N-hydroxysuccinimide (NHS) ester; a protected amine can be useful for preventing aldehyde crosslinking in the resulting construct.

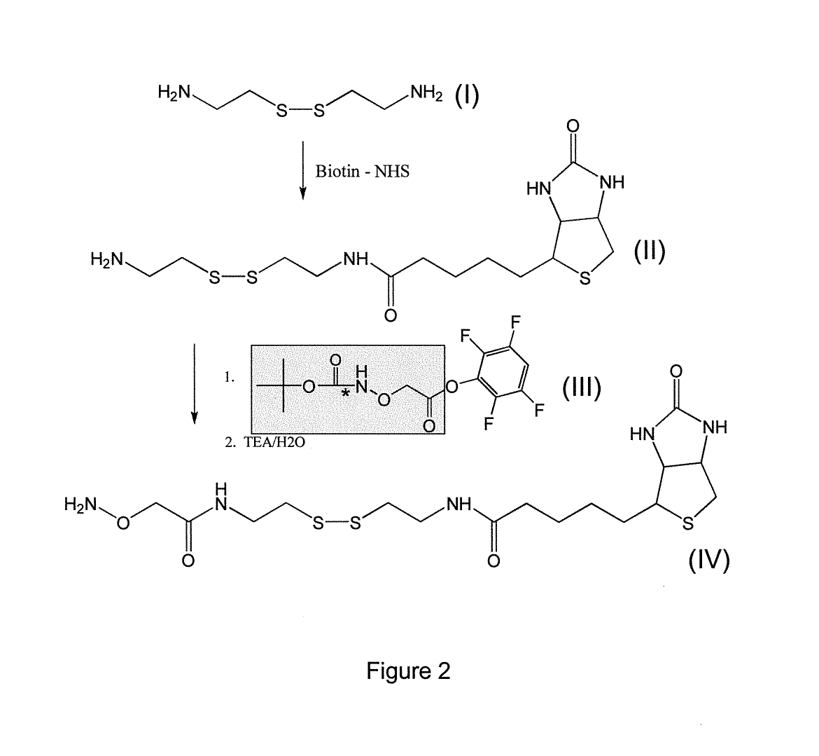

In preferred embodiments, an affinity tag (e.g., biotin, digoxigenin, etc.) is added to a linker prior to the linker being added to a molecule to be enriched. An exemplary method for synthesizing a cleavable biotin affinity tag is provided in FIG. 2. The linker (I) is combined with an affinity tag linked to NHS ester (Biotin-NHS) to produce the linker-affinity tag construct (II). This construct is then combined with another molecule (III) in a two-step reaction to add an aldehyde reactive end, which can be reacted with a nucleic acid modification to link the affinity tag thereto. This reactive group is especially suitable for linkage to a modification comprising an oxidized sugar (e.g., oxidized glucose-5-hmC). The first step is addition of the portion of compound III shown in a grey box; the second step is a treatment to cleave the bond between the amine and the carboxyl group (*). (Although molecule III is shown with a C(CH₃)₃ group, in alternative embodiments this group is instead a CH₃ group.) The product of this two-step reaction is shown as molecule (IV), which comprises three main components: an affinity tag (in this example, biotin), an aldehyde reactive end, and a disulfide bond subject to cleavage. Cleavage of the disulfide bond allows removal of the affinity tag after enrichment.

Selected examples of reactive functionalities useful for associating an affinity tag to a binding partner in a solid phase are shown in Table I, wherein a covalent bond results from such a reaction. Those of skill in the art will know of other bonds suitable for use in the present invention.

TABLE I

| | Complementary | |
|---|---|---|
| Reactive functionality | group | The resulting bond |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides/imides |
| aryl halides | thiols | thiophenols |
| aryl halides | amines | aryl amines |
| aziridines | thiols | thioethers |
| boronates | glycols | boronate esters |
| carboxylic acids | amines/anilines | carboxamides |
| carboxylic acids | alcohols | esters |
| carboxylic acids | hydrazines | hydrazides |
| carbodiimides | carboxylic acids | N-acylureas or anhydrides |
| diazoalkanes | carboxylic acids | esters |
| epoxides | thiols (amines) | thioethers (alkyl amines) |
| epoxides | carboxylic acids | esters |
| haloacetamides | thiols | thioethers |
| haloplatinate | amino | platinum complex |
| haloplatinate | heterocycle | platinum complex |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonyl halides | amines/anilines | sulfonamides |

### IV. Selection of Hemi-Modified Loci

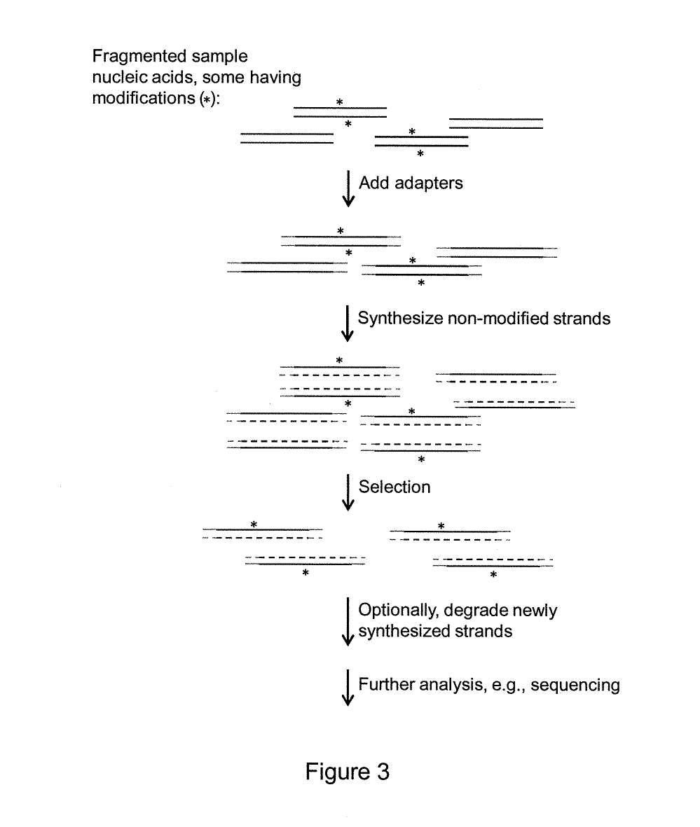

In certain aspects of the instant invention, methods for detecting modified nucleotides within a double-stranded nucleic acid molecule involve conversion of modified loci into hemi-modified loci and subsequent selection with an agent (e.g., enzyme, antibody, etc.) that recognizes hemi-modified loci. Such agents include maintenance methyltransferases, e.g., DNMT1 and PpMET1, which specifically recognize hemi-methylated sequences. For example, a sample comprising methylated loci can be fragmented into linear double-stranded nucleic acid fragments. Ligation of adapter sequences to the ends of the fragments, denaturation, and addition of primer sequences complementary to the adapter sequences provides a priming site for a polymerase enzyme to begin synthesis of a nascent strand using unmethylated nucleotides. The resulting double-stranded nucleic acid products are hemi-methylated at loci that comprised a methylated base in the original sample. An affinity-tagged maintenance methyltransferase (MMTase) is provided under reaction conditions that favor binding to the hemi-methylated sites, and the affinity tags are used to separate the nucleic acids bound by the MMTase, e.g. by binding the tags to an immobilized binding partner and washing away the unbound nucleic acids. The bound, hemi-methylated nucleic acids are released from the MMTases, e.g., by heating or otherwise changing the reaction conditions to increase the off rate. The resulting pool of nucleic acids is enriched for hemi-methylated molecules that can be subsequently sequenced. FIG. 3 illustrates an exemplary embodiment of the general strategy in which adapters are added, strands not comprising modifications are synthesized to convert the modified double-stranded fragments into hemi-modified double-stranded fragments, and the nascent strands can be optionally removed prior to further analysis.

In certain preferred aspects, additional stem-loop adapter sequences are subsequently added to the selected hemi-modified nucleic acids such that the 3′ and 5′ termini at each end are linked together. This produces a nucleic acid molecule that is topologically closed such that denaturation or other separation of the double-stranded portion results in a single-stranded circular molecule. These closed molecules are preferred templates for polymerase-mediated sequencing-by-synthesis reactions since they allow a polymerase enzyme to repeatedly sequence both strands of a double-stranded template in a “rolling-circle” mode to generate redundant sequencing data for each strand. For more information on generation of redundant sequencing data, see U.S. Pat. Nos. 7,476,503 and 8,153,375, both of which are incorporated herein by reference in their entireties for all purposes. In some preferred embodiments, the stem-loop adapters are added to all double-stranded nucleic acid products of the first polymerase extension reaction prior to enrichment. This strategy can increase the efficiency of the stem-loop ligation reaction. Once the ligation is complete, the resulting set of closed molecules having stem-loop adapters is subjected to the enrichment process to select those molecules having hemi-modified sites, and only the hemi-modified molecules are retained for subsequent analysis, e.g., sequencing. It will be understood that reference to methylated and hemi-methylated sites is merely exemplary and that other types of modifications can be analyzed by the methods herein. Likewise, although maintenance methyltransferases are one example of a protein that specifically binds hemi-modified sites, other agents can also be used depending on the type of modification under investigation, e.g., antibodies, DNA-repair proteins/enzymes, etc.

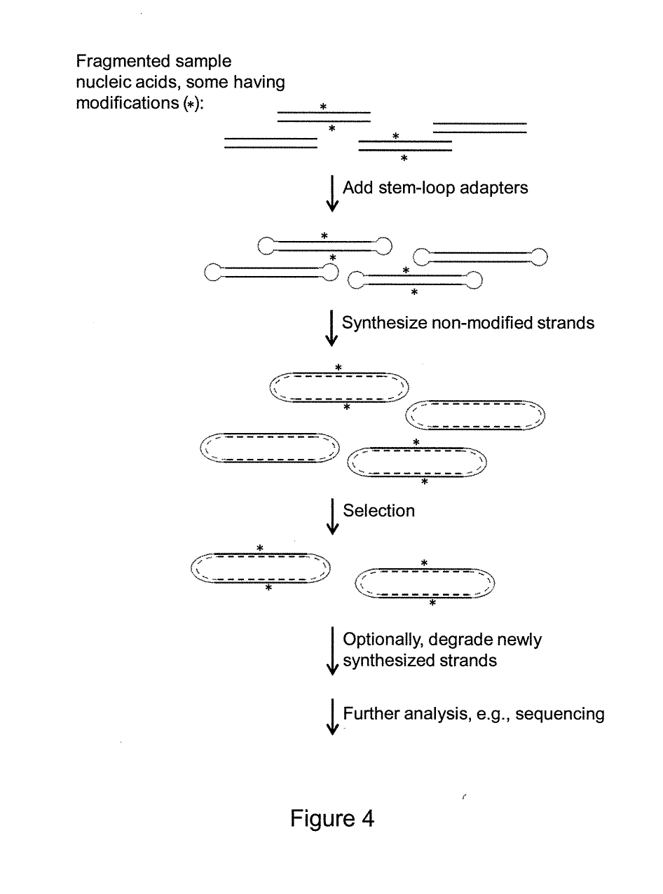

In certain embodiments, the molecules to be sequenced are heat-denatured to allow binding of fresh primers to only the strands from the original sample. For example, the original fragments can be treated to block the activity of an exonuclease used to degrade the nascent, non-modified strand. As above, the adapter-containing fragments are subsequently denatured, and primer sequences complementary to the adapter sequences provide priming sites for a synthesis of anascent, non-modified strands. The hemi-modified molecules resulting from synthesizing a non-modified strand opposite a modified strand are subjected to an enrichment to select hemi-modified molecules and remove molecules that are fully non-modified, and since the nascent strand is susceptible to degradation by the exonuclease, it can be removed after enrichment to provide a mixture of single-stranded, modified templates for sequencing. For single-strand-specific exonucleases, the hemi-modified molecules are typically denatured prior to exonuclease treatment. Exonucleases that are suitable for degrading single-stranded nucleic acids are well known in the art and include RecJf (5′->3′ polarity), ExoI (3′->5′ polarity), and ExoT (3′->5′ polarity). Alternatively, the hemi-modified double-stranded fragments need not be denatured if the exonuclease is capable of degrading a single strand of a double-stranded nucleic acid, e.g., T7 exonuclease (5′->3′ polarity) and ExoIII (3′->5′ polarity). Since these and other select exonucleases have difficulty cleaving phosphorothioate linkages, in certain embodiments the adapters added to the original double-stranded fragments (supra) comprise phosphorothioate linkages to protect the original strand from degradation. Protection can also be afforded by blocking the ends of the original (e.g., modified) strands, e.g., by phosphoryl or acetyl groups, or by bulky chemical groups. The hemi-modified duplexes would therefore have one strand protected (original or “native” strand) and a second strand that is nuclease-susceptible (nascent strand). Treatment with the exonuclease would therefore degrade only the nascent strand, leaving the original strand available for subsequent sequence analysis, e.g., by additional of new primer sequences and subsequent sequencing by synthesis. FIG. 4 illustrates an exemplary embodiment of the general strategy in which stem-loop adapters are added, strands not comprising modifications are synthesized to convert the modified, single-stranded, circular constructs into hemi-modified double-stranded constructs, which can be subjected to a selection process prior to optionally degrading the nascent strands prior to further analysis.

In other preferred embodiments, a sample comprising modified loci is fragmented and hairpin or stem-loop adapters are ligated to the termini of the resulting double-stranded fragments. This produces a closed nucleic acid that, when the two strands of the double-stranded fragment are separated, becomes a single-stranded circular nucleic acid molecule. Synthesis and use of such closed nucleic acids are provided, e.g., in U.S. Pat. No. 8,153,375. A primer is added and allowed to hybridize to the closed nucleic acid molecule, e.g., within the single-stranded portion of one of the adapters or within a denatured region of the double-stranded portion. A polymerase binds to the primer-template complex and synthesizes a nascent strand complementary to the nucleic acid in the presence of non-modified nucleotides by extending the primer at the 3′ terminus. Opposite a modified base in the original nucleic acid molecule, the polymerase will incorporate a non-modified base, thereby creating a hemi-modified site that comprises one strand corresponding to the original closed nucleic acid and comprising the modification, and a second, nascent and unmodified strand. For example, opposite a 5-MeC or 5-hmC nucleotide in the original nucleic acid molecule, the polymerase will incorporate a non-modified G nucleotide, since the binding specificity of 5-MeC and 5-hmC is the same as that of a C nucleotide. Where the polymerase is a non-strand-displacing polymerase and only one primer is bound to the template, the polymerase proceeds around the template to generate a complementary strand, stopping synthesis when it encounters the 5′ end of the original primer with which synthesis was initiated. At that point, the nascent strand will have a gap. Double-stranded products having hemi-modified sites, e.g., where the original strand had a modified base and the nascent strand does not, can be enriched as described above. Subsequent to enrichment, a strand-displacing polymerase can be used to reinitiate synthesis, e.g., during a sequencing by synthesis reaction. Where the polymerase requires a larger single-stranded region for initiation than the gap left by the non-strand-displacing polymerase, the gap can be enlarged by limited exonuclease degradation, which is routine in the art. In some embodiments, the nascent strand can be degraded entirely to provide the original closed molecule comprising a double-stranded fragment of the original sample (which contains at least one modification based upon being selected during the enrichment) and two stem-loop adapters. Addition of a primer, e.g., complementary to an adapter, primes the template for subsequent sequencing-by-synthesis.

In certain embodiments, the same polymerase can be used to both generate the hemi-modified molecules and to perform the subsequent sequencing reaction after enrichment. For example, a strand-displacing polymerase can be used where the adapter sequences comprise elements that cause the polymerase to pause but not dissociate. The enrichment procedure is performed, and the nascent strand synthesis is reinitiated for the sequencing portion of the analysis. The elements that cause a pause in polymerase activity can be bulky groups that are removable, e.g., via mild chemical or photo-induced cleavage, binding sites for proteins that bind to the template tightly enough to resist displacement by the polymerase, or abasic sites that can be repaired prior to reinitiation of polymerization. Further examples of elements that can cause pausing of polymerase activity are described in detail in U.S. Pat. No. 7,901,889 and U.S. Patent Publication No. 20110195406, both of which are incorporated herein by reference in their entireties for all purposes.

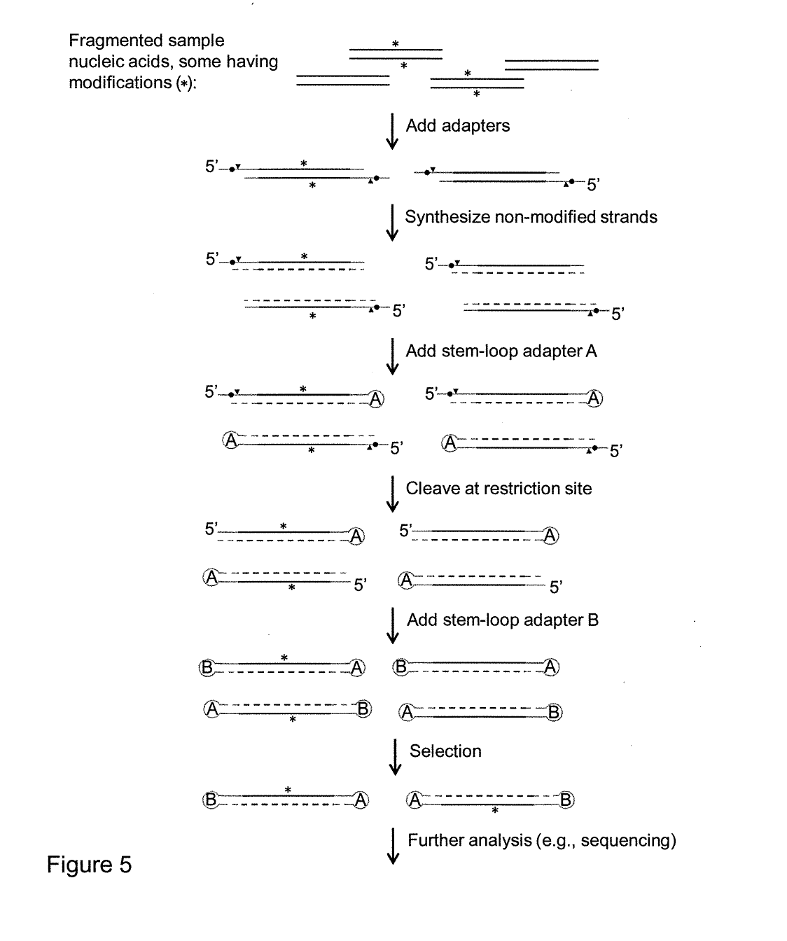

In some embodiments, specially designed adapter sequences provide a means of attaching different stem-loop sequences to each end of a nucleic acid fragment to be analyzed. One such adapter is a double-stranded adapter that is blunt-ended at one terminus and has a 5′-overhang at the opposite terminus. FIG. 5 illustrates an exemplary embodiment of the general strategy in which adapters are added and used to synthesize complementary strands not comprising modifications to convert the modified double-stranded fragments into hemi-modified double-stranded fragments. The double-stranded adapter illustrated has a polymerase-blocking group () within the 5′-overhang near but outside of the double-stranded portion of the adapter, and also comprises one strand of a restriction endonuclease recognition site (▾) within the single-stranded portion, between the double-stranded portion and the blocking group. Two adapters are linked to each fragment, one at each end, with the blunt ends of the adapters ligated to the ends of the fragment. This arrangement provides a 5′-overhang at each end of the nucleic acid product. The fragments are denatured and primers complementary to the double-stranded portion of the adapters are introduced in the presence of a polymerase that extends the primers to create hemi-modified double-stranded molecules where the original fragments had a modified site. Since the overhang comprises the blocking group (e.g., phosphorothioate linkages, abasic sites, etc.) that does not permit polymerase synthesis the synthesis terminates prematurely resulting double-stranded molecules comprising a 5′-overhang at one end and a blunt terminus at the other. The 5′-overhang is somewhat shorter than that of the original adapter because the polymerization continues up to the blocking group, which renders the restriction endonuclease site double-stranded and, therefore, susceptible to cleavage by an appropriate restriction endonuclease. Further, the strand having the 5′-overhang is also the original strand from the sample, which has the modification if one is present. A first stem-loop adapter (A) having a blunt terminus is ligated to the blunt ends of these fragments, but the 5′-overhang prevents ligation to the opposite end. Following ligation and removal of the excess, unligated first stem-loop adapters, the 5′-overhang is removed by treatment with a restriction endonuclease that cleaves at the double-stranded restriction endonuclease recognition site (▾). Once the 5′-overhang is removed, a second stem-loop adapter (B) is linked to the terminus. The second stem-loop adapter may comprise a terminus with an overhang complementary to that left by the endonuclease treatment. Alternatively, the end of the fragment can be blunted, e.g., by exonuclease treatment prior to ligation of a blunt-ended second stem-loop adapter (B). In either case, the resulting construct comprises two different stem-loop adapters, one at each end. This pool of constructs is subsequently selected for hemi-modified sequences and further analyzed, e.g., by sequencing.

These constructs are particularly useful for sequencing-by-synthesis applications because they allow the practitioner to choose which strand to sequence first. If the sequencing primer is complementary to the first stem-loop adapter, then the first strand sequenced will be the strand from the original nucleic acid sample; if the sequencing primer is complementary to the second stem-loop adapter, then the first strand sequenced will be the unmodified nascent strand. In certain embodiments, the constructs can be used to produce partially double-stranded circular molecules that can be subjected to a selection based on binding to a modification in a single-stranded portion of the construct, e.g., by an antibody linked to an affinity tag. For example, where a primer is added that is complementary to the second stem-loop adapter and the first stem-loop adapter has a feature that causes the polymerase to pause, a polymerization reaction will generate a construct having a double-stranded portion comprising to the nonmodified strand and a single-stranded portion comprising the modified strand. A binding agent specific for the modification in a single-stranded form is introduced under conditions that favor binding to select only those constructs that have the modification and the constructs that do not bind (are modification-free) are removed. The remaining constructs are released from the binding agent (e.g., antibody) and are subjected to further analysis, e.g., after treatment to remove or repair the pause site. In some embodiments a new primer is added to initiate a subsequent sequencing reaction, and in other embodiments the 3′ end of the newly generated strand is used for this purpose. In further embodiments, the pause site can prevent passage of a first polymerase used to generate the partially double-stranded construct, but does not prevent passage of a second polymerase subsequently used to sequence the construct.

Agents that selectively bind hemi-modified loci are preferably linked or linkable to a solid surface, e.g., a bead or column. In the case of methylated nucleotides, an affinity-tagged maintenance methyltransferase that can bind tightly to hemi-methylated sites, e.g., in the absence of a cofactor necessary for the methylation of the hemi-methylated site, can be used to pull down those molecules. Nucleic acids that do not contain such hemi-methylated sites remain in solution and can be removed, e.g., by washing or buffer exchange. The methyltransferase can then be removed, either by chemical treatment, or by providing the missing cofactor, the latter of which will also transform the hemi-methylated sites into fully methylated sites. The resulting pool of nucleic acids molecules is enriched for modified nucleic acids, which can then be subjected to further analysis, e.g., sequencing, cloning, etc.

In other embodiments, rather than using a polymerase to generate a hemi-modified site in the nucleic acid, a primer that is complementary to a known motif (consensus sequence for the modification, e.g., methylation) can be annealed to the closed nucleic acid, which is optionally denatured prior to addition of the primer. In some embodiments, the primer comprises a structure that favors hybridization with the closed nucleic acid over intramolecular hybridization, such as those described for driver nucleic acids above, e.g., O-methyl nucleotides, LNA, etc. Since the primer does not contain the modification, binding of it to a site that does contain the modification provides the hemi-modified region needed for binding of the agent that selectively binds hemi-modified loci.

Depending upon the concentration of the nucleic acid sample to be subjected to the enrichment procedures described herein, it may be beneficial to add “carrier” nucleic acids that do not comprise the modification of interest to enhance the ligase reaction that joins the stem-loop adapter(s) to the ends of a fragment to be sequenced. Ligation can be inefficient where the amount of nucleic acids present is too low. By addition of carrier nucleic acids, the concentration of the nucleic acid sample is raised to increase the efficiency of the ligation reaction. Ironically, addition of carrier effectively “un-enriches” the sample for the target region prior to the enriching procedure, however, can result in production of a more enriched sample by the end of the procedure. In preferred embodiments, the carrier nucleic acids added are preferably lacking recognition sites for any endonucleases used to generate cuts flanking a nucleic acid region comprising the modification of interest. In some embodiments, these carrier nucleic acids are linked to affinity tags to allow their efficient removal from the nucleic acid sample once there is no more need for a higher nucleic acid concentration. In some embodiments, the carrier nucleic acids lack an affinity tag that is bound to fragments comprising the modification, so that they are not captured with the modified nucleic acids and can be removed with other non-modified nucleic acids during the enrichment procedure.

Different types of carrier nucleic acids are known and used in the art, e.g., DNA from lambda phage, plasmid DNA, synthetic oligonucleotides, etc.

### V. Selection of Sugar-Modified Nucleotides

In certain aspects of the instant invention, methods for detecting sugar-modified nucleotides using proteins that selectively bind sugar moieties that are linked to the nucleotides. For example, various methods for adding a glucose moiety to a 5-hmC nucleotide are provided, e.g., in U.S. Patent Publication No. 2011/0301045; Josse, et al. (1962) J. Biol. Chem. 237:1968; and Lariviere, et al. (2004) J. Biol. Chem. 279(33):34715-20, all of which are incorporated herein by reference in their entireties for all purposes. Methods for linking glucose-modified hmC to a biotin tag to facilitate enrichment have been described, e.g., in Pastor, et al. (2011) Nature 473(7347):394-397; and Song, et al. (2011) Nature Biotechnology 29:68-72, both of which are incorporated herein by reference in their entireties for all purposes.

The present invention provides alternatives to the methods provided in the art that utilize sugar-binding moieties, e.g., glucose-binding proteins, to enrich a sample for nucleic acid molecules comprising sugar-modified nucleotides. For example, lectins are sugar-binding proteins that are highly specific for their sugar moieties. Concanavalin A and other commercially available lectins have been used in affinity chromatography for purifying glycol-proteins. The invention provides the insight that these proteins can also be used to isolate and enrich nucleic acids having sugar moieties specifically recognized by a particular lectin. Table II provides an exemplary listing of lectins contemplated for use with the methods and compositions described herein.

TABLE II

| | | | |
|---|---|---|---|
| Lectin | | | |
| Symbol | Lectin Name | Source | Ligand Motif |

Mannose binding lectins

| | | | |
|---|---|---|---|
| ConA | Concanavalin A | Canavalia | α-D-mannosyl and α-D-glucosyl residues |
| | | ensiformis | branched α-mannosidic structures (high α- |
| | | | mannose type, or hybrid type and biantennary |
| | | | complex type N-Glycans) |
| LCH | Lentil lectin | Lens culinaris | Fucosylated core region of bi- and |
| | | | triantennary complex type N-Glycans |
| GNA | Snowdrop lectin | Galanthus | α 1-3 and α 1-6 linked high mannose |
| | | nivalis | structures |

Galactose/N-acetylgalactosamine binding lectins

| | | | |
|---|---|---|---|
| RCA | Ricin, Ricinus | Ricinus | Galβ1-4GlcNAcβ1-R |
| | communis | communis | |
| | agglutinin, | | |
| | RCA120 | | |
| PNA | Peanut agglutinin | Arachis | Galβ1-3GalNAcα1-Ser/Thr (T-Antigen) |
| | | hypogaea | |
| AIL | Jacalin | Artocarpus | (Sia)Galβ1-3GalNAcα1-Ser/Thr (T-Antigen) |
| | | integrifolia | |
| VVL | Hairy vetch lectin | Vicia villosa | GalNAcα-Ser/Thr (Tn-Antigen) |

N-acetylglucosamine binding lectins

| | | | |
|---|---|---|---|
| WGA | Wheat germ | Triticum | GlcNAcβ1-4GlcNAcβ1-4GlcNAc, Neu5Ac |
| | agglutinin | vulgaris | (sialic acid) |

N-acetylneuraminic acid binding lectins

| | | | |
|---|---|---|---|
| SNA | Elderberry lectin | Sambucus nigra | Neu5Acα2-6Gal(NAc)-R |
| MAL | Maackia amurensis | Maackia | Neu5Ac/Gcα2,3Galβ1,4Glc(NAc) |
| | leukoagglutinin | amurensis | |
| MAH | Maackia amurensis | Maackia | Neu5Ac/Gcα2,3Galβ1,3(Neu5Acα2,6)GalNac |
| | hemoagglutinin | amurensis | |

Fucose binding lectins

| | | | |
|---|---|---|---|
| UEA | Ulex europaeus | Ulex europaeus | Fucα1-2Gal-R |
| | agglutinin | | |
| AAL | Aleuria aurantia | Aleuria aurantia | Fucα1-2Galβ1-4(Fucα1-3/4)Galβ1-4GlcNAc, |
| | lectin | | R2-GlcNAcβ1-4(Fucα1-6)GlcNAc-R1 |

Use of sugar-binding proteins, such as lectins or others, facilitates enrichment of sugar-modified nucleic acid molecules. For example, these proteins can be attached to beads or a chromatography column, e.g., via an affinity tag. After addition of the sugar moieties to the nucleic acid molecules, the mixture is exposed to the sugar-binding protein, which binds only to those fragments comprising the sugar moieties. This exposure can occur while the sugar-binding protein is attached to a solid surface (e.g., bead or column), or attachment of the protein can occur subsequent to binding to the sugar-modified nucleic acids. Once the sugar-modified nucleic acids are bound to the sugar-binding proteins attached to the solid surface, the free nucleic acids that do not comprise sugar moieties and are therefore not bound to the sugar-binding proteins can be removed, e.g., by washing or buffer exchange. The removed nucleic acids may be discarded, or retained for further analysis.

Similarly, anticarbohydrate antibodies specific to a sugar moiety can be used to enrich a mixture of nucleic acids for those comprising the sugar moiety to which the antibody binds. As described above for lectins, the sugar-binding antibodies can be immobilized on beads, columns, or another solid surface to allow removal of the nucleic acids not containing the sugar moiety to provide a nucleic acid mixture enriched for nucleic acids comprising the sugar moiety.

Yet further, aptamers have been shown to specifically bind sugar moieties, and these can also be used in such enrichment strategies. (See, e.g., Yang, et al. (1998) Proc. Natl. Acad. Sci. USA 95(10): 5462-5467, which is incorporated herein by reference in its entirety for all purposes.) Aptamers are high affinity nucleic acid ligands that can comprise DNA or RNA, and those with a specificity for a given sugar moiety can be selected from a random pool of nucleic acids sequences. One selection procedure, termed SELEX (systemic evolution of ligands by exponential) enrichment, involves the iterative isolation of ligands out of the random sequence pool with affinity for a defined target molecule and PCR-based amplification of the selected RNA or DNA oligonucleotides after each round of isolation. Aptamer affinities compare favorably to characterized protein affinities for simple sugars, and more complex monomers could well provide significantly increased affinity while remaining selective for only particular combinations of monomer units. For example, there has been extensive development of RNA aptamers capable of interacting with aminoglycoside antibiotics, which contain both amino and hydroxyl groups for hydrogen bonding interactions and three or more ring structures. See, e.g., Wang, et al. (1995) Chem. Biol. 2:281-290; Lato, et al. (1995) Chem. Biol. 2:291-303; Wallis, et al. (1995) Chem. Biol. 2:543-552; and Jiang, et al. (1997) Chem. Biol. 4:35-50, all of which are incorporated herein by reference in their entireties for all purposes. As such, in certain aspects of the invention, sugar moieties that are more complex than simple monosaccharides, e.g., diglucose, etc., are added to a subset of nucleic acids (e.g., those comprising a modification to which the sugar can be selectively linked). The nucleic acids to which these complex sugar moieties have been added are then selected by addition of an agent that selectively binding the complex sugar moiety, such as a lectin, antibody, or aptamer. The agent-nucleic acids complexes are separated from nucleic acids not bound by the agent to provide a nucleic acid mixture enriched for nucleic acids comprising the sugar moiety, e.g., by immobilizing the agent upon a solid surface, as described elsewhere herein.

### VI. Enrichment by Exonuclease Degradation

Certain aspects of the invention take advantage of an organism's restriction modification system to enrich a genomic sample from that organism for nucleic acids having a modification. Restriction modification systems are commonly found in prokaryotic organisms and function to distinguish “self”′ nucleic acids from “foreign” nucleic acids, such as those of bacteriophages that infect bacterial cells. Sequence-specific endonucleases produced by the bacteria degrade the foreign nucleic acids while sparing the bacteria's own nucleic acids. In order to prevent destruction of its own nucleic acids, the sequences corresponding to the endonucleases are modified so that they are not recognized and therefore not cleaved. This modification is typically addition of a methyl group, which does not interfere with the base-pairing specificity, but the modified sequence is not recognized by the endonuclease.

In certain embodiments, methods are provided for enriching a genomic sample for modified nucleic acids by degrading non-modified nucleic acids with a sequence-specific nuclease that cannot cleave the sequence if it comprises a given modification, e.g., methylation. For example, a genomic sample is isolated from an organism suspected of having methylated bases at a known sequence. The sample is fragmented and the ends of the resulting fragments are capped by ligation of hairpin or stem-loop adapters to both ends. The capped fragments are treated with an endonuclease that only cleaves the specific sequence if it does not contain the modification, and subsequent treatment with an exonuclease degrades the fragments that were so cleaved. The remaining fragments either do not comprise the specific sequence, or comprise the specific sequence with the modification that prevents cleavage. These fragments can be further analyzed, e.g., to sequence them and map the modified sequences.

In alternative embodiments, the hairpin-ligated fragments (or stem-loop-ligated fragments) can be divided into multiple pools, and each pool treated with a different modification-sensitive endonuclease prior to exonuclease degradation. In doing so, the sample can be analyzed to determine the modification status of a set of different defined sequences, since each pool will be enriched for fragments resistant to a different endonuclease. Further, where a modified sequence of interest is located in the same fragment as an unmodified sequence, the fragment will only be degraded in one of the pools, i.e., the pool being treated by the endonuclease specific for the unmodified sequence. As such, the modification will be able to be further analyzed in another of the pools in which that fragment is not degraded.

Many different modification-sensitive restriction endonucleases are known and commercially available, e.g., from New England Biolabs, and are contemplated for use in these enrichment strategies. Alternatively or in addition, such restriction endonucleases could be used for the initial fragmentation of the sample nucleic acids. Since they only cleave at unmodified sites, this would ensure that the fragmentation would not disrupt any modified sites, rendering them undetectable in the subsequent analysis.

VII. Further Modifications that Facilitate Enrichment and Detection

In certain aspects of the invention, modifications to be analyzed can be further modified prior to the analysis, e.g., to facilitate enrichment and/or detection. Methods for further modifying modified nucleic acids are provided, e.g., in Josse, et al. (1962) J. Biol. Chem. 237:1968; Lariviere, et al. (2004) J. Biol. Chem. 279(33):34715-20; Pastor, et al. (2011) Nature 473(7347):394-397; Song, et al. (2011) Nature Biotechnology 29:68-72; U.S. Patent Publication No. 20110183320; U.S. Patent Application No. 61/637,687, filed Apr. 24, 2012; U.S. Patent Publication No. 20110301045; and U.S. Patent Publication No. 20110236894, all of which are incorporated herein by reference in their entireties for all purposes. Such methods include, but are not limited to, adding chemical modifications and/or affinity tags to a base modification, binding agents (e.g., proteins) directly to the base modifications, and enzymatically modifying the base modifications.

In some embodiments of the present invention, a nucleic acid sample is fragmented and exposed to a solid surface (e.g., beads, a column, etc.) having a moiety bound thereto that is chemically or enzymatically linkable to the modified bases present in a subset of the nucleic acid fragments where linkage of the moiety provides a further modification to the modified base. During the exposure, conditions are provided that promote linkage of any fragments having the modification to the moiety on the solid surface, and once the linkage has been made all fragments not linked to the surface (i.e., those not having the modification) are removed. Subsequent to removal of the unbound nucleic acid fragments, the moiety is removed from the solid surface such that it remains linked to the modification, thereby providing the further modification. In preferred embodiments, the moiety bound to the modification enhances detection of the modification in a subsequent analysis. For example, where the modified base is a 5-hmC nucleotide, the moiety bound to the solid surface can be a sugar group, e.g., glucose. When a fragment comprising 5-hmC is removed from the solid surface it retains the sugar moiety, which facilitated detection of the modified base in subsequent sequencing, e.g., by enhancing a kinetic signal during real-time, single-molecule sequencing-by-synthesis as further described in U.S. Patent Publication No. 20110183320. In certain preferred embodiments, the solid surface is not only used to retain and isolate the modified nucleic acids, but also as a vehicle to deliver the modified nucleic acids to a reaction site for further analysis. For example, the solid surface can be a magnetic bead that is guided by a magnetic field to one or more reaction sites configured to accept the modified nucleic acids.

In other embodiments, modifications are introduced, e.g., chemically or enzymatically, to unmodified bases in a nucleic acid sample to further distinguish them (e.g., in sequencing data) from the modified bases that were already present in the sample. For example, any method for enriching the nucleic acid population for nucleic acids having a modification of interest can be used, e.g., those described elsewhere herein. After the enrichment process, the remaining nucleic acids (i.e., those comprising at least one modified base) are treated to modify nucleotides having the same nitrogenous base but not having the modification. For example, where 5-mC-containing fragments are selected, a moiety can be linked to all canonical C nucleotides in the population, where no such moiety is added to the 5-mC nucleotides. This moiety enhances the difference between signals corresponding to 5-mC and C nucleotides, thereby aiding in distinction between the two different nucleotides during a subsequent analysis. In certain embodiments, a methyltransferase that binds to C nucleotides but not 5-mC nucleotides is used. Preferably, the methyltransferase is added in the absence of a cofactor needed to add a methyl group to the C nucleotides, such that the methyltransferase remains bound to the C nucleotides. The presence of the methyltransferase on C nucleotides in the template during sequencing provides a detectable signal that is distinct from those of 5-mC nucleotides (as well as A, T, or G nucleotides). As such, the methyltransferase improves distinction between the bases and thereby enhances accuracy of the sequencing reaction.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations, e.g., sequentially or simultaneously. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

### VIII. Applications

The enriched compositions described herein are particularly useful in nucleic acid sequencing reactions, e.g., polymerase mediated, template dependent synthesis of nucleic acids, which can be observed using real-time techniques for a variety of desired goals, including in particular, determination of information about the template sequence. A number of methods have been proposed for determination of sequence information using incorporation of fluorescent or fluorogenic nucleotides into the synthesized strand by a DNA or other polymerase, and the compositions of the invention are applicable to these methods. While several of these methods employ iterative steps of nucleotide introduction, washing, optical interrogation, and label removal, preferred uses of these compositions utilize “real-time” determination of incorporation. Such methods are described in detail in, for example, U.S. Pat. Nos. 7,056,661, 7,052,847, 7,033,764 and 7,056,676, the full disclosures of which are incorporated herein by reference in their entirety for all purposes.

Briefly, such methods observe an immobilized polymerase/template/primer complex as it incorporates labeled nucleotide analogs. Using optical techniques that illuminate small volumes around the complex with excitation radiation, e.g., TIRF methods, optical confinements like Zero Mode Waveguides (ZMWs) (See, U.S. Pat. Nos. 6,917,726, 7,013,054, 7,181,122, 7,292,742 and 7,170,050 and 7,302,146), and the like, one can identify incorporation events based upon the optical signature of their associated fluorophore, as compared to non incorporated, randomly diffusing labeled nucleotide analogs. By providing each different type of nucleotide with a distinguishable fluorescent label, e.g., having a distinguishable emission spectrum, one can identify each base as it is incorporated, and consequently read out the sequence of the template as the nascent strand is created against it. By utilizing the compositions of the invention, negative impacts of the fluorescent label on the polymerase or other components of the labeled complex (See, e.g., published U.S. Patent Application No. 2007/0161017), can be reduced or eliminated by moving the label portion away from the reactant portion and consequently, the active site of the enzyme, or other sensitive portions of the complex.

In some embodiments, the methods herein are used to enrich target nucleic acids from complex samples, e.g., metagenomic samples. Metagenomic samples include, but are not limited to, environmental samples such as soil, water, and air; agricultural samples such as produce and meat; industrial samples such as generated waste; and biological samples such as forensic collections and bacterial mixtures. The methods are especially beneficial where the target nucleic acids are a minority species in a mixture of nucleic acids. For example, where the sample is blood collected from an infected human individual the enrichment can separate human nucleic acids from “non-human” nucleic acids that may be present, e.g., by capturing the known human nucleic acids and separating them from the non-human nucleic acids. The isolated non-human nucleic acids can be subsequently analyzed to determine their source, e.g., one or more pathogenic organisms. Similarly, where it is desired to determine whether a sample comprises a particular minority species, the minority species can be specifically captured, isolated from the rest of the nucleic acids in the sample, and subsequently detected.

In certain aspects, the methods herein allow a modification profile to be generated for a nucleic acid sample that comprises a count and specific mapping of modified loci. Such a profile is extremely difficult with traditional methods that use MeDIP to pull down methylated nucleic acids since MeDIP methods suffer from bias when comparing singly-methylated fragments with multiply-methylated fragments, with the singly-methylated fragments captures with a much lower efficiency than the multiply-methylated fragments. Further, the methods provided herein allow single-molecule sequencing that includes base-by-base detection of multiple modifications within a single template nucleic acid. As such, the modifications are accurately mapped at a single nucleotide resolution rather than simply identifying that “somewhere” in a fragment there is a modification. Although MeDIP can be combined with bisulfite sequencing, this involves sequencing copies of the template in two separate sequencing reactions: “+bisulfite” and “−bisulfite” sequencing. Since bisulfite treatment is harsh, it can also introduce mutations into the template nucleic acids, further confounding analysis of the sequence of the original sample nucleic acids. The present methods provide a modification profile across a template nucleic acid that includes identification of not only the selected modifications, but also other modifications that are present in the sample nucleic acids near enough to the selected modifications to have been included in the sequencing templates generated from the enriched pool of fragments.

### IX. Kits

The compositions of the invention are optionally provided in kit form, including various components of an overall analysis in combination with instructions for carrying out the desired analysis. In particular, such kits typically include the compositions of the invention, including at least one, but preferably multiple types of labeled nucleotide analogs of the invention, e.g., A, T, G and C analogs. Each of the different types of labeled nucleotide analogs in the kit will typically comprise a distinguishable labeling group, as set forth above. In addition to the analog compositions, the kits will optionally include one or more components of a polymerase complex, including, for example polymerase enzymes, such as any of a number of different types of strand displacing polymerase enzymes. Examples of such polymerases include, e.g., phi29 derived polymerases, and the polymerase enzymes described in, e.g., Published International Patent Application Nos. WO 2007/075987, WO 2007/075873 and WO 2007/076057, the full disclosures of which are incorporated herein by reference in their entirety for all purposes.

Additional reaction components are also optionally included in such kits, such as buffers, salts, universal priming sequences for initiation of synthesis, and the like. In addition, in particularly preferred aspects, the kits of the invention will typically include a reaction substrate that includes reaction regions for carrying out and observing the synthesis reactions for identification of sequence information. Such substrates include, e.g., multi-well micro or nano plates, as well as arrayed substrates, e.g., planar transparent arrays that include discrete reaction regions defined by, e.g., structural, chemical or other means. For example, patterned arrays of complexes may be provided disposed upon planar transparent substrates for observation. Alternatively and preferably, the substrate component comprises an array or arrays of optically confined structures like zero mode waveguides. Examples of arrays of zero mode waveguides are described in, e.g., U.S. Pat. No. 7,170,050, the full disclosure of which is incorporated herein by reference in its entirety for all purposes.

Although described in some detail for purposes of illustration, it will be readily appreciated that a number of variations known or appreciated by those of skill in the art may be practiced within the scope of present invention. All terms used herein are intended to have their ordinary meaning unless an alternative definition is expressly provided or is clear from the context used therein. To the extent any definition is expressly stated in a patent or publication that is incorporated herein by reference, such definition is expressly disclaimed to the extent that it is in conflict with the ordinary meaning of such terms, unless such definition is specifically and expressly incorporated herein, or it is clear from the context that such definition was intended herein. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes.

What is claimed is:

1. A method of generating a pool of sequencing templates enriched for a type of modified base, the method comprising:
a) fragmenting a nucleic acid sample comprising the type of modified base, thereby generating a mixture comprising a first subset of nucleic acid fragments comprising the type of modified base and a second subset of nucleic acid fragments that do not comprise the type of modified base;
b) linking adapters to nucleic acid fragments in both the first subset and the second subset; and
c) retaining the nucleic acid fragments in the first subset from the mixture, thereby generating a pool of sequencing templates enriched for the type of modified base.

2. The method of claim 1, wherein the retaining comprises binding the type of modified base to an agent linked to an affinity tag.

3. The method of claim 1, wherein the type of modified base is a methylated cytosine.

4. The method of claim 1, wherein the type of modified base is a hydroxymethylated cytosine.

5. The method of claim 1, wherein the adapters are hairpin or stem-loop adapters that link 3′ and 5′ termini at each end of the nucleic acid fragments.

6. The method of claim 5, wherein the adapters added to a first end of the nucleic acid fragments in the first subset are different from the adapters added to a second end of the nucleic acid fragments in the first subset.

7. The method of claim 1, wherein the retaining comprises affinity purifying the nucleic acid fragments.

8. The method of claim 1, wherein said retaining comprises binding driver nucleic acids to the first subset of nucleic acid fragments comprising the type of modified base.

9. The method of claim 8, wherein the driver nucleic acids are complementary to a sequence motif that comprises the modified base in the first subset of nucleic acid fragments.

10. The method of claim 8, wherein the driver nucleic acids are generated from an aliquot of the nucleic acid sample.

11. The method of claim 10, wherein the aliquot is subjected to cleavage with a methyl-dependent restriction endonuclease.

12. The method of claim 11, further comprising subjecting the aliquot to a size selection following the cleavage and prior to the binding.

13. The method of claim 11, further comprising subjecting the aliquot to an amplification reaction following the cleavage and prior to the binding.

14. The method of claim 11, wherein the methyl-dependent restriction endonuclease is MspJI.

15. The method of claim 8, wherein the driver nucleic acids comprise an affinity tag.

16. The method of claim 8, wherein the driver nucleic acids comprise a sequence motif suspected of comprising the type of modified base in the first subset of nucleic acid fragments.

17. The method of claim 8, wherein the binding of the driver nucleic acids to the first subset of nucleic acid fragments is performed in the presence of a strand-exchange protein.

18. The method of claim 1, wherein the nucleic acid fragments in both the first subset and the second subset are not amplified prior to the retaining.

19. The method of claim 1, further comprising removing second subset of nucleic acid fragments that do not comprise the type of modified base.

20. The method of claim 19, wherein the removing comprises nuclease digestion.
